(19) United States
(12) Patent Application Publication
(10) Pub. No.: US 2011/0028318 A1
(43) Pub. Date: Feb. 3, 2011

(54) HERBICIDAL COMPOUNDS BASED ON N-AZINYL-N'-PYRIDYLSULPHONYLUREAS

(75) Inventors: Klaus-Helmut Mueller, Duesseldorf (DE); Ernst Rudolf Gesing, Erkrath-Hochdahl (DE); Christian Waldraff, Bad Vilbel (DE); Heînz Kehne, Hofheim (DE); Dieter Feucht, Eschborn (DE); Jeffrey Martin Hills, Idstein (DE); Christopher Hugh Rosinger, Hofheim (DE); Jan Dittgen, Frankfurt am Main (DE)

Correspondence Address: Baker Donelson Bearman, Caldwell & Berkowitz, PC, 920 Massachusetts Ave, NW, Suite 900, Washington, DC, 20001, US

(73) Assignee: Bayer CropScience AG, Monheim (DE)

(21) Appl. No.: 12/809,416

(22) PCT Filed: Dec. 4, 2008
(86) PCT No.: PCT/EP08/10265

§ 371 Date: Jun. 18, 2010

(30) Foreign Application Priority Data
Dec. 20, 2007 (EP) 07024727.5
Jan. 25, 2008 (DE) 10 2008 006 005.4

(51) Int. Cl. A01N 25/32; C07D 401/14; C07D 403/14; C07D 413/04; A01N 47/36; A01P 13/02

(52) U.S. Cl. 504/103; 544/320; 544/209; 544/212; 546/272.1; 504/215; 504/136

(57) ABSTRACT

There are described compounds of the formula (I)

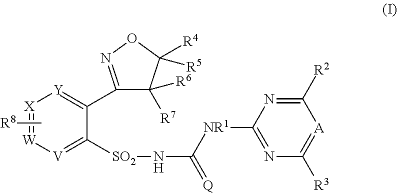

in which the respective substituents have the meanings given in the description. The compounds of the formula (I) can be used as herbicides and plant growth regulators.

The present invention relates to N-azinyl-N′-pyridylsulfonylureas. The present invention furthermore relates to mixtures of the abovementioned urea derivatives with other herbicides and/or safeners. Moreover, the present invention relates to processes for the preparation of the abovementioned urea derivatives and to the use of these compounds as herbicides and plant growth regulators alone and in admixture with safeners and/or in admixture with other herbicides, in particular to their use for controlling plants in specific plant crops or as plant growth regulators.

It has already been disclosed that certain N-azinyl-N′-arylsulfonylureas with single open-chain hydroxamic ester groups in the aryl moiety such as, for example, N-(4,6-dimethylpyrimidin-2-yl)-N′-(2-methoxyaminocarbonylphenylsulfonyl)urea and the corresponding N′-(2-n-octyloxyaminocarbonylphenylsulfonyl)urea have herbicidal properties (cf. DE 3 516 435 A, EP 0 173 958 A, U.S. Pat. No. 4,704,158).

Furthermore, there are also known certain herbicidally active N-azinyl-N′-hetarylsulfonylureas which are substituted in the hetaryl moiety by O,N-dialkylated, likewise open-chain hydroxamic acid groups (cf. EP 0 301 784 A); however, corresponding cyclic hydroxamic acid derivatives have not been described to date.

The active substances which are already known from the above-mentioned specifications have disadvantages when used, be it
(a) that they have no or only else an insufficient herbicidal activity against harmful plants,
(b) that only an unduly narrow spectrum of harmful plants can be controlled, or
(c) that they have an unduly narrow selectivity in crops of useful plants.

It is therefore desirable to provide alternative chemical active substances based on corresponding urea derivatives which can be employed as herbicides or plant growth regulators and with which certain advantages in comparison with the prior-art systems are associated.

As a result, the object of the present invention is in general to provide corresponding alternative urea derivatives which can be employed as herbicides or plant growth regulators, in particular with a satisfactory herbicidal activity against harmful plants, a broad spectrum for harmful plants and/or a high selectivity in crops of useful plants. In this context, these urea derivatives should preferably feature a better profile of characteristics, in particular a better herbicidal activity against harmful plants, a broader spectrum for harmful plants and/or a higher selectivity in crops of useful plants than the urea derivatives known from the prior art.

There have now been novel N-azinyl-N′-pyridylsulfonylureas of the formula (I),

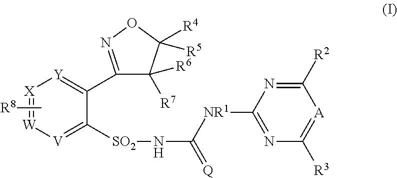

in which
V, W, X and Y are chosen such that one of these indices represents nitrogen and the remaining indices represent carbon atoms, which may be unsubstituted or else substituted by the radical R⁸ shown;
A is selected from the group consisting of nitrogen and CR⁹; where
   R⁹ is selected from the group consisting of hydrogen, alkyl, halogen and haloalkyl;
R¹ is selected from the group consisting of hydrogen and an optionally substituted radical from the series consisting of alkyl, alkoxy, alkoxyalkyl, alkenyl, alkynyl, cycloalkyl, cycloalkylalkyl, aralkyl and aryl;
R² is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino;
R³ is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino,
R⁴ to R⁷, in each case independently of one another, are selected from the group consisting of hydrogen, halogen, cyano, alkyl, alkoxy, alkylthio, alkylsulfinyl, alkylsulfonyl, alkylamino, dialkylamino, alkylcarbonyl, alkoxycarbonyl, alkylaminocarbonyl or dialkylaminocarbonyl, it being possible for the radicals to be unsubstituted or to have attached to them one or more radicals selected from the group consisting of halogen, cyano, alkoxy and alkylthio, or R⁴ and R⁶, and R⁵ and R⁷, respectively, represent an alkylidene group which is optionally interrupted by oxygen or sulfur,
R⁸ is selected from the group consisting of hydrogen, halogen, cyano, thiocyanato, nitro, alkyl, alkoxy, alkylthio, alkylsulfinyl, alkylsulfonyl, alkylamino, dialkylamino, alkylcarbonyl, alkoxycarbonyl, alkylaminocarbonyl, or dialkylaminocarbonyl, it being possible for the radicals to be unsubstituted or to have attached to them one or more radicals selected from the group consisting of halogen, cyano, alkoxy and alkylthio,
Q is selected from the group consisting of oxygen or sulfur, in particular oxygen,
   and salts of compounds of the formula (I).

A first embodiment of the present invention comprises compounds of the formula (I) in which
A is preferably selected from the group consisting of nitrogen and CH.

A second embodiment of the present inventions comprises compounds of the formula (I) in which
R¹ is preferably selected from the group consisting of hydrogen, alkyl, alkoxy, alkoxyalkyl, alkenyl and alkynyl, it being possible for the radicals to be unsubstituted or to have attached to them one or more halogen atoms,
R¹ is especially preferably selected from the group consisting of hydrogen, methyl, ethyl, methoxy, methoxymethyl and ethoxy,
R¹ is particularly preferably selected from the group consisting of hydrogen and methyl,
   and
R¹ is specifically preferably hydrogen.

A third embodiment of the present invention comprises compounds of the formula (I) in which
R² is preferably selected from the group consisting of hydrogen, halogen, alkyl, alkoxy, alkylthio, alkylamino and dialkylamino, it being possible for the radicals to be unsubstituted or to have attached to them one or more halogen atoms,
R² is especially preferably selected from the group consisting of hydrogen, chlorine, methyl, ethyl, trifluoromethyl, methoxy, ethoxy, trifluoroethoxy, difluoromethoxy, methylthio, methylamino and dimethylamino,
   and
R² is specifically preferably selected from the group consisting of hydrogen, chlorine, methyl, methoxy, methylthio and dimethylamino.

A fourth embodiment of the present invention comprises compounds of the formula (I) in which
R³ is preferably selected from the group consisting of hydrogen, halogen, alkyl, alkoxy, alkylthio, alkylamino and dialkylamino, it being possible for the radicals to be unsubstituted or to have attached to them one or more halogen atoms,
R³ is especially preferably selected from the group consisting of hydrogen, chlorine, methyl, ethyl, trifluoromethyl, methoxy, ethoxy, trifluoroethoxy, difluoromethoxy, methylthio, methylamino and dimethylamino,
   and
R³ is specifically preferably selected from the group consisting of methyl, methoxy and trifluoroethoxy.

A fifth embodiment of the present invention comprises compounds of the formula (I) in which
R⁴ to R⁷, in each case independently of one another, are preferably selected from the group consisting of hydrogen, halogen, cyano, alkyl, alkoxy, alkylthio, alkylsulfinyl, alkylsulfonyl, alkylamino, dialkylamino, alkylcarbonyl, alkoxycarbonyl, alkylaminocarbonyl; or dialkylamino, it being possible for the radicals to be unsubstituted or to have attached to them one or more halogen atoms,
R⁴ to R⁷, in each case independently of one another, are especially preferably selected from the group consisting of hydrogen, fluorine, chlorine, cyano, methyl, ethyl, propyl, isopropyl, cyclopropyl, n-, i-, s- or tert-butyl, methylthio, methylsulfinyl, methylsulfonyl, methoxycarbonyl and ethoxycarbonyl, it being possible for the radicals to be unsubstituted or to have attached to them one or more halogen atoms, and R⁴ and R⁶ and/or R⁵ and R⁷ represent a CH₂—CH₂—CH₂— group, a CH₂—CH₂—CH₂—CH₂— group, a CH₂—O—CH₂— group, a CH₂—S—CH₂— group, a CH₂—O—CH₂—CH₂— group, a CH₂—CH₂—O—CH₂— group or a CH₂—CH₂—O—CH₂—CH₂— group,
   and
R⁴ to R⁷, in each case independently of one another, are specifically preferably selected from the group consisting of hydrogen, methyl, ethyl, propyl and isopropyl, or R⁴ and R⁶ and/or R⁵ and R⁷ represent a CH₂—CH₂—CH₂— group, a CH₂—CH₂—CH₂—CH₂— group, a CH₂—O—CH₂— group, a CH₂—S—CH₂— group, a CH₂—O—CH₂—CH₂— group, a CH₂—CH₂—O—CH₂— group or a CH₂—CH₂—O—CH₂—CH₂— group.

A sixth embodiment of the present invention comprises compounds of the formula (I) in which
R⁸ is preferably selected from the group consisting of halogen, cyano, thiocyanato, nitro, alkyl, alkoxy, alkylthio, alkylsulfinyl, alkylsulfonyl, alkylamino, dialkylamino, alkylcarbonyl, alkoxycarbonyl, alkylaminocarbonyl and dialkylaminocarbonyl, it being possible for the radicals to be unsubstituted or to have attached to them one or more halogen atoms,
R⁸ is especially preferably selected from the group consisting of fluorine, chlorine, bromine, iodine, cyano, methyl, methoxy, ethoxy, methylthio, ethylthio, methylsulfinyl, ethylsulfinyl, methylsulfonyl, ethylsulfonyl, methylamino and dimethylamino, it being possible for the radicals to be unsubstituted or to have attached to them one or more halogen atoms,
   and
R⁸ is specifically preferably hydrogen.

Within the scope of these embodiments of the present invention, it is possible to combine the individual general, preferred and especially preferred meanings for the substituents R¹ to R⁸, Q and A as desired. This means that the present invention comprises compounds of the formula (I) in which for example the substituent R¹ has a preferred meaning and the substituents R² to R⁸ have the general meanings, or else for example that the substituent R² has a preferred meaning, the substituent R³ an especially preferred meaning, and the remaining substituents have the general meanings. These individual combinations are not mentioned expressly for reasons of clarity, but must be considered as being comprised by the present invention.

The heterocycle of the compounds of the formula (I)

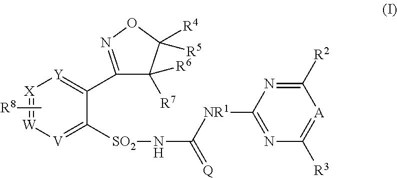

which is linked directly to the dihydroisoxazole ring has a nitrogen atom, i.e. a radical selected from the group consisting of V, W, X and Y corresponds to a nitrogen atom. The remaining three radicals have the meaning of a carbon atom, it being possible for the carbon atoms optionally to be substituted by the radical R⁸.

In a special embodiment of the present invention, the compound of the formula (I) therefore has the following structure (Ia), in which V has the meaning of a nitrogen atom:

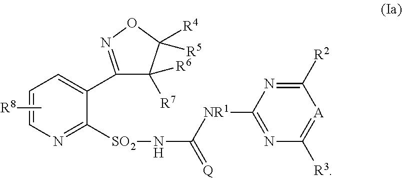

In yet a further embodiment of the present invention, the compound of the formula (I) therefore has the following structure (Ib), in which W has the meaning of a nitrogen atom:

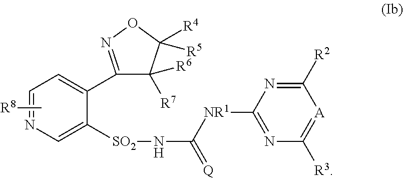

In yet a further embodiment of the present invention, the compound of the formula (I) therefore has the following structure (Ic), in which X has the meaning of a nitrogen atom:

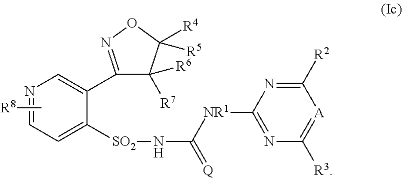

In yet a further embodiment of the present invention, the compound of the formula (I) therefore has the following structure (Id), in which Y has the meaning of a nitrogen atom:

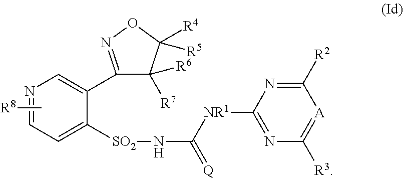

In each of these four embodiments, the individual radicals R¹ to R⁸, A and Q can have the above-defined general, preferred and especially preferred meanings.

Especially preferred among these four embodiments are according to the invention those compounds in which V, W and Y have the meaning nitrogen (compounds of the formula (Ia), (Ib), (Id)).

Particularly preferred are those compounds of the formula (I), in which V and Y have the meaning nitrogen (compounds of the formula (Ia), (Id)).

Most particularly preferred are those compounds of the formula (I), in which V has the meaning nitrogen (compounds of the formula (Ia)).

In the compounds of the formula (I), the substituents and radicals R¹ to R⁸, Q and A have the above general, preferred, especially preferred, particularly preferred and very particularly preferred meanings.

The present invention preferably also relates to the lithium, sodium, potassium, magnesium, calcium, ammonium, C₁-C₄-alkylammonium, di(C₁-C₄-alkyl)ammonium, tri(C₁-C₄-alkyl)ammonium, tetra(C₁-C₄-alkyl)ammonium, tri(C₁-C₄-alkyl)sulfonium, C₅- or C₆-cycloalkylammonium, di(C₁-C₂-alkyl)benzylammonium and tri(C₁-C₂-alkyl)benzylammonium salts of compounds of the formula (I) in which R¹ to R⁸, A and Q have the above general, preferred, especially preferred and particularly preferred meanings and which can be prepared by generally customary methods.

In addition, the compounds of the formula (I) can where appropriate form salts by addition reaction of a suitable inorganic or organic acid, such as, for example, HCl, HBr, H₂SO₄ or HNO₃, but also oxalic acid or sulfonic acids, onto a basic group such as, for example, amino or alkylamino. Suitable substituents which are present in deprotonated form, such as, for example, sulfonic acids or carboxylic acids, can form internal salts with groups which can be protonated in turn, such as amino groups. Salts can also be formed by replacing, in the case of suitable substituents such as, for example, sulfonic acids or carboxylic acids, the hydrogen by a cation which is suitable for the agrochemical sector. Examples of these salts are metal salts, in particular alkali metal salts or alkaline earth metal salts, in particular sodium and potassium salts, or else ammonium salts, salts with organic amines or quaternary ammonium salts with cations of the formula [NRR′R″R′″]⁺, in which R to R′″ in each case independently of another represent an organic radical, in particular alkyl, aryl, aralkyl or alkylaryl.

In the formula (I) and all remaining formulae in the present invention, the radicals alkyl, alkoxy, haloalkyl, haloalkoxy, alkylamino, alkylthio, haloalkylthio, alkylsulfinyl, alkylsulfonyl, haloalkylsulfinyl and haloalkylsulfonyl and the corresponding unsaturated and/or substituted radicals in the carbon skeleton can in each case be straight-chain or branched. Unless otherwise specified, the lower carbon skeletons, for example those with 1 to 6 carbon atoms, in particular 1 to 4 carbon atoms, or, in the case of unsaturated groups, having 2 to 6 carbon atoms, in particular 2 to 4 carbon atoms, are preferred among these radicals. Alkyl radicals, also in the composite meanings such as alkoxy, haloalkyl and the like, are, for example, methyl, ethyl, propyls such as n- or i-propyl, butyls such as n-, iso- or tert-butyl, pentyls such as n-pentyl, isopentyl or neopentyl, hexyls such as n-hexyl, i-hexyl, 3-methylpentyl, 2,2-dimethylbutyl or 2,3-dimethylbutyl, heptyls such as n-heptyl, 1-methylhexyl or 1,4-dimethylpentyl; alkenyl and alkynyl radicals have the meaning of the unsaturated radicals which are possible and which correspond to the alkyl radicals and which comprise at least one double bond or triple bond, preferably one double bond or triple bond. Alkenyl is, for example, vinyl, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl; alkynyl is, for example, ethynyl, propargyl, but-2-yn-1-yl, but-3-yn-1-yl and 1-methylbut-3-yn-1-yl.

Examples of cycloalkyl groups are cyclopropyl, cyclobutyl, cyclopentyl, cyclohexyl, cycloheptyl and cyclooctyl. The cycloalkyl groups may be present in bicyclic or tricyclic form.

If haloalkyl groups and haloalkyl radicals of haloalkoxy, haloalkylthio, haloalkenyl, haloalkynyl and the like are specified, the lower carbon skeletons, for example those having 1 to 6 carbon atoms or 2 to 6, in particular 1 to 4, carbon atoms or preferably 2 to 4 carbon atoms, and the corresponding unsaturated and/or substituted radicals in the carbon skeleton are in each case straight-chain or branched in these radicals. Examples are difluoromethyl, 2,2,2-trifluoroethyl, trifluoroallyl, 1-chloroprop-1-yl-3-yl. The term “halo” is used synonymously with “halogen” according to the invention.

Alkylene groups in these radicals are the lower carbon skeletons, for example those having 1 to 10 carbon atoms, in particular 1 to 6 carbon atoms or preferably 2 to 4 carbon atoms (unless otherwise defined) and the corresponding unsaturated and/or substituted radicals in the carbon skeleton which can in each case be straight-chain or branched. Examples are methylene, ethylene, n- and isopropylene and n-, sec-, iso- and tert-butylene.

Hydroxyalkyl groups in these radicals are the lower carbon skeletons, for example those having 1 to 6 carbon atoms, in particular 1 to 4 carbon atoms, and the corresponding unsaturated and/or substituted radicals in the carbon skeleton which can in each case be straight-chain or branched. Examples are 1,2-dihydroxyethyl and 3-hydroxypropyl.

Halogen is fluorine, chlorine, bromine or iodine. Haloalkyl, haloalkenyl and haloalkynyl are alkyl, alkenyl or alkynyl which are partially or fully substituted by halogen, preferably by fluorine, chlorine or bromine, in particular by fluorine and/or chlorine, for example monohaloalkyl, perhaloalkyl, CF₃, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; the same applies analogously to haloalkenyl and other halogen-substituted radicals.

Aryl is a mono-, bi- or polycyclic aromatic system, for example phenyl or naphthyl, preferably phenyl.

Unless otherwise defined, the definition “substituted by one or more radicals” means one or more identical or different radicals.

The substituents mentioned by way of example (“first substituent level”) can, if they contain hydrocarbon-comprising moieties, optionally be further substituted therein (“second substituent level”), for example by one of the substituents as defined for the first substituent level. Corresponding further substituent levels are possible. Preferably, the term “substituted radical” only comprises one or two substituent levels.

Preferred in the case of radicals with carbon atoms are those with 1 to 6 carbon atoms, preferably 1 to 4 carbon atoms, in particular 1 or 2 carbon atoms. As a rule, preferred are substituents from the group consisting of halogen, for example fluorine and chlorine, (C₁-C₄)-alkyl, preferably methyl or ethyl, (C₁-C₄)-haloalkyl, preferably trifluoromethyl, (C₁-C₄)-alkoxy, preferably methoxy or ethoxy, (C₁-C₄)-haloalkoxy, nitro and cyano.

When an aryl radical is substituted, this may preferably be phenyl which is monosubstituted or polysubstituted, preferably up to trisubstituted, by identical or different radicals selected from the group consisting of halogen, (C₁-C₄)-alkyl, (C₁-C₄)-alkoxy, (C₁-C₄)-haloalkyl, (C₁-C₄)-haloalkoxy, cyano and nitro, for example o-, m- and p-tolyl, dimethylphenyls, 2-, 3- and 4-chlorophenyl, 2-, 3- and 4-trifluoromethyl and 2-, 3- and 4-trichloromethylphenyl, 2,4-, 3,5-, 2,5- and 2,3-dichlorophenyl, o-, m- and p-methoxyphenyl.

Depending on the nature and linkage of the substituents, the compounds of the formula (I) may be present as stereoisomers. The stereoisomers which are possible, such as enantiomers, diastereomers, Z and E isomers, which are defined by their specific spatial shape, are all comprised by formula (I).

If, for example, one or more alkenyl groups are present, then diastereomers (Z and E isomers) may occur. If, for example, one or more asymmetric carbon atoms are present, then enantiomers and diastereomers may occur. Stereoisomers may be obtained by customary separation methods from the mixtures which the preparation gives rise to. Likewise, stereoisomers may be prepared selectively by using stereoselective reactions, using optically active starting materials and/or adjuvants. The invention therefore also relates to all stereoisomers which are comprised by the formula (II), but not specified in their specific stereomeric form, and to mixtures of these.

### Preparation of the Compounds of the Formula (I) According to the Invention

The present invention furthermore relates to processes for the preparation of corresponding compounds of the formula (I) and/or their salts.

In a first embodiment of the present invention, the compounds of the formula (I) are prepared by reacting (4,5-dihydroisoxazol-3-yl)pyridinesulfonamides of the formula (II)

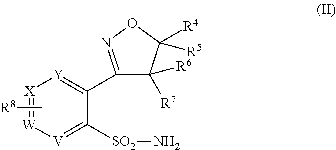

with a heterocyclic (thio)carbamate of the formula (III)

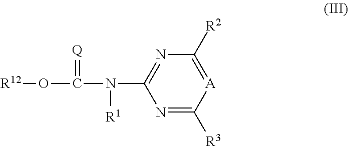

in which R¹² is a substituted or unsubstituted (C₁-C₂₀)-hydrocarbon radical such as aryl or alkyl, preferably optionally substituted phenyl or optionally substituted (C₁-C₄)-alkyl, and in which V, W, X, Y, R¹ to R⁸, Q and A have the abovementioned meanings.

In this context, the compounds of the formula (II) can be obtained by reacting the compounds of the formula (X) with a chlorinating agent such as chlorine gas and ammonia solution as shown in the reaction scheme hereinbelow:

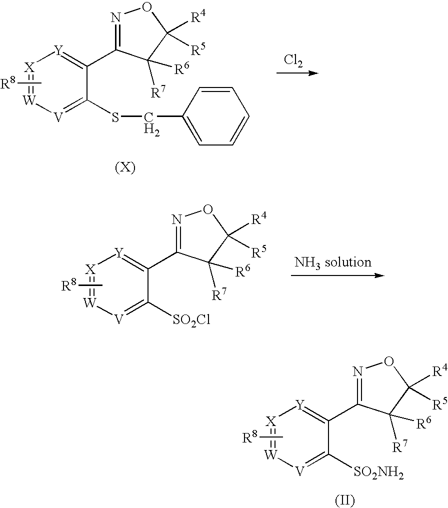

The compounds of the formula (X), in turn, can be obtained by reacting compounds of the formula (XI) with benzyl mercaptan as shown in the reaction scheme hereinbelow starting from the compounds of the formula (XI):

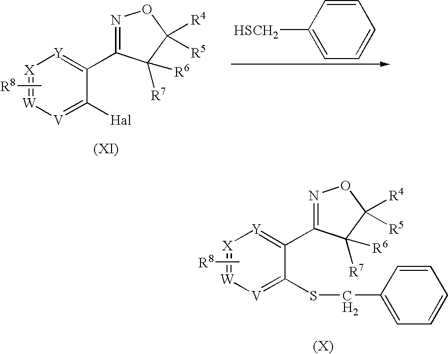

The compounds of the formula (XI), in turn, can be obtained by closing the ring and forming the heterocycle, starting from compounds of the formula (XII) as shown in the reaction scheme hereinbelow:

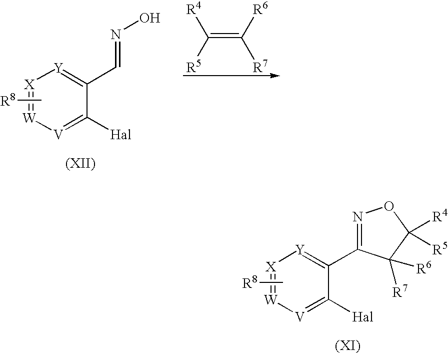

2-Chloronicotinic aldehyde oxime and substituted 2-chloronicotinic aldehyde oximes are known, for example, from the prior art and can be prepared by methods known to the skilled worker (cf. WO 2003/090539, Helvetica Chimica Acta 59, 211-21 (1976), EP 0 012 158).

In a second embodiment of the present invention, the compounds of the formula (I) are prepared by reacting (4,5-dihydroisoxazol-3-yl)pyridinesulfonyl iso(thio)cyanates of the formula (IV)

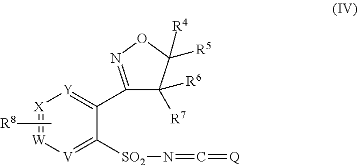

with an amino heterocycle of the formula (V)

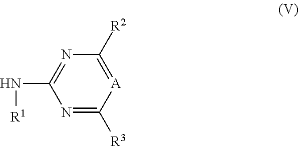

in which V, W, X, Y, R¹ to R⁸, Q and A have the above meanings.

In a third embodiment of the present invention, the compounds of the formula (I) are prepared by reacting sulfonyl (thio)carbamates of the formula (VI)

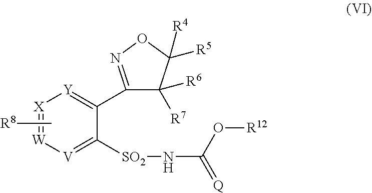

in which R¹² is a substituted or unsubstituted (C₁-C₂₀)-hydrocarbon radical such as aryl or alkyl, preferably optionally substituted phenyl or optionally substituted (C₁-C₄)-alkyl, with an amino heterocycle of the formula (V)

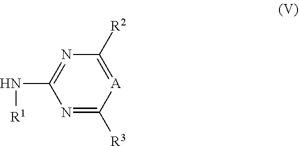

in which V, W, X, Y, R¹ to R⁸, Q and A have the abovementioned meanings.

In a fourth embodiment of the present invention, the compounds of the formula (I) are prepared by reacting (4,5-dihydroisoxazol-3-yl)pyridinesulfonamides of the formula (II)

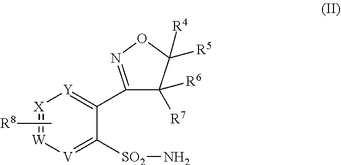

with an iso(thio)cyanate of the formula (VII)

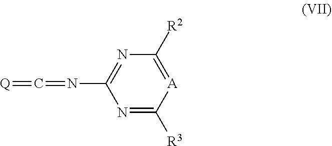

in which R¹ is hydrogen and R² to R⁸, V, W, X, Y, Q and A have the abovementioned meanings, optionally in the presence of a reaction auxiliary.

In a fifth embodiment of the present invention, the compounds of the formula (I) are prepared by initially reacting, with base catalysis, an amino heterocycle of the formula (V)

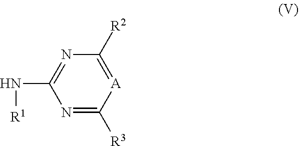

with a carbonic ester, for example diphenyl carbonate, and reacting, in a one-pot reaction, the resulting intermediate of the formula (III)

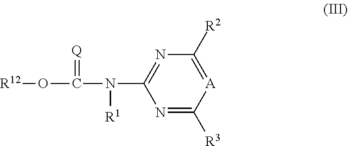

with a (4,5-dihydroisoxazol-3-yl)pyridinesulfonamide of the formula (II)

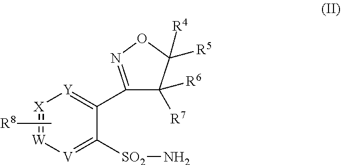

(cf. JP1989221366), in which V, W, X, Y, R¹ to R⁸, Q and A have the abovementioned meanings.

In a sixth embodiment of the present invention, the compounds of the formula (I) are prepared by reacting (4,5-dihydroisoxazol-3-yl)pyridinesulfonyl halides of the formula (VIII)

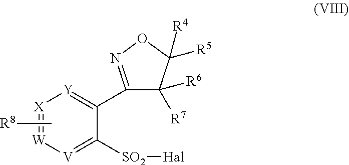

in which Hal is a halogen atom, preferably chlorine, with a (thio)cyanate, in particular a metal (thio)cyanate, in particular an alkali metal (thio)cyanate, such as sodium (thio)cyanate, to give an iso(thio)cyanate of the formula (IV)

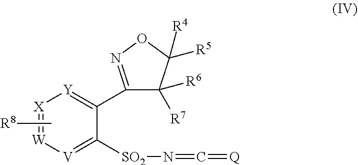

or a solvated (stabilized) derivative thereof, and subsequently with an amino heterocycle of the formula (V)

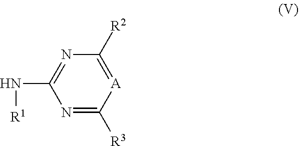

in which V, W, X, Y, R¹ to R⁸, Q and A have the abovementioned meanings.

In a seventh embodiment of the present invention, the compounds of the formula (I) where Q=oxygen are prepared by reacting (4,5-dihydroisoxazol-3-yl)pyridinesulfonamides of the formula (II)

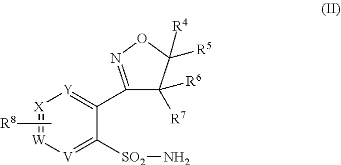

with a heterocyclic biscarbamate of the formula (IX),

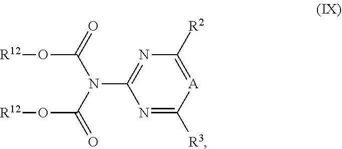

in which R¹² is a substituted or unsubstituted (C₁-C₂₀)-hydrocarbon radical such as aryl or alkyl, preferably optionally substituted phenyl or optionally substituted (C₁-C₄)-alkyl, in the presence of a basic reaction auxiliary, where V, W, X, Y, R¹ to R⁸ and A have the abovementioned meanings.

In an eighth embodiment of the present invention, the compounds of the formula (I) are prepared by initially reacting, with base catalysis (4,5-dihydroisoxazol-3-yl)pyridinesulfonamides of the formula (II)

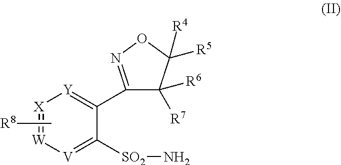

with a carbonic ester, for example diphenyl carbonate, and reacting, in a one-pot reaction, the resulting intermediate of the formula (VI)

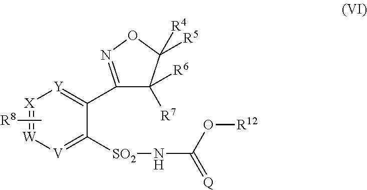

with an amino heterocycle of the formula (V)

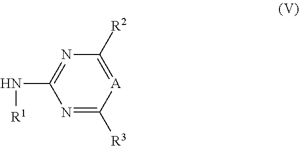

in which V, W, X, Y, R¹ to R⁸, Q and A have the abovementioned meanings.

All these processes lead to compounds of the formula (I) according to the invention.

Inert solvents are used in each case in each of the abovementioned process variants. For the purposes of the present invention, inert solvents are understood as meaning those which are inert under the respective reaction conditions, i.e. which, in particular, do not react with the starting materials, but which need not be inert under all reaction conditions.

Examples of organic solvents which can be used within the scope of the present invention are aromatic or aliphatic solvents such as benzene, toluene, xylene, mesitylene, hexane, heptane, octane, cyclohexane; aliphatic and aromatic halohydrocarbons such as methylene chloride, dichloroethane, chloroform, carbon tetrachloride, chlorobenzene, dichlorobenzene, ethers such as diethyl ether, dibutyl ether, diisobutyl ether, methyl tert-butyl ether, isopropyl ethyl ether, diisopropyl ether, tetrahydrofuran, and dioxane; furthermore also dimethyl sulfoxide and acid amide derivatives, such as N,N-dimethylformamide, N,N-dimethylacetamide and N-methyl-2-pyrrolidone, and also carboxylic acid esters such as ethyl acetate, or else diglymes, dimethyl glycol; nitriles such as acetonitrile, propionitrile or butyronitrile, and also ketones such as acetone, methyl ethyl ketone or cyclohexanone. Especially preferred are toluene, xylene, dichlorobenzene, chlorobenzene, acetonitrile, acetone, butyronitrile or ethyl acetate. However, the present invention is not limited to the solvents mentioned above by way of example.

The reaction temperature at which the reactions according to the above embodiments can be carried out may vary within wide ranges. For example, the reactions can be carried out at a temperature of from 0 to 100° C., preferably from 20 to 70° C.

In general, the reactions of the present inventions are carried out under atmospheric pressure. However, they may also be carried out under elevated pressure or reduced pressure—in general between 0.1 bar and 10 bar.

The processes for the preparation of the N-azinyl-N′-pyridylsulfonylureas of the formula (I) according to the invention are, where necessary, carried out in the presence of a basic reaction auxiliary.

Suitable reaction auxiliaries are all customary inorganic or organic bases. These include, for example, the hydrides, hydroxides, amides, alkoxides, acetates, carbonates or hydrogencarbonates of alkali metals or alkaline earth metals, such as, for example, lithium hydride, sodium hydride, potassium hydride, calcium hydride, lithium amide, sodium amide, potassium amide, sodium methoxide, potassium methoxide, sodium ethoxide, potassium ethoxide, sodium propoxide, potassium propoxide, aluminum isopropoxide, sodium tert-butoxide, potassium tert-butoxide, sodium hydroxide, potassium hydroxide, ammonium hydroxide, sodium acetate, potassium acetate, calcium acetate, ammonium acetate, sodium carbonate, potassium carbonate, calcium carbonate, ammonium carbonate, sodium hydrogencarbonate, potassium hydrogencarbonate, and basic organic nitrogen compounds such as trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl- and 4-methylpyridine, 2,4-dimethyl-, 2,6-dimethyl-, 3,4-dimethyl- and 3,5-dimethylpyridine, 5-ethyl-2-methylpyridine, N-methylpyridine, 4-(N,N-dimethylamino)pyridine, diazabicyclooctane (DABCO), diazabicyclononene (DBN) or diazabicycloundecene (DBU).

### Intermediates

Also subject matter of the present application are certain intermediates which are generated according to the above-shown synthetic routes when preparing the compounds of the formula (I) according to the invention.

In a first embodiment of the intermediates, accordingly, the present invention also comprises compounds of the formula (II)

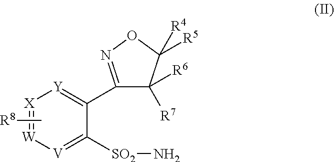

in which the radicals V, W, X, Y, R⁴, R⁵, R⁶, R⁷ and R⁸ have the general, preferred and especially preferred meanings already indicated further hereinabove.

In a second embodiment of the intermediates, further subject matter of the present invention are also compounds of the formula (IV)

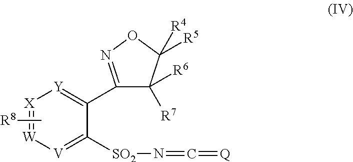

in which the radicals V, W, X, Y, R⁴, R⁵, R⁶, R⁷, R⁸ and Q have the general, preferred and especially preferred meanings already indicated further hereinabove.

In a third embodiment of the intermediates, further subject matter of the present invention are also compounds of the formula (X)

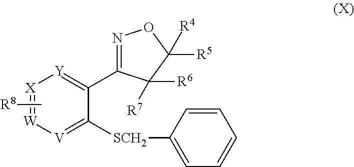

in which the radicals V, W, X, Y, R⁴, R⁵, R⁶, R⁷ and R⁸ have the general, preferred and especially preferred meanings already indicated further hereinabove.

In a fourth embodiment of the intermediates, further subject matter of the present invention are also compounds of the formula (XI)

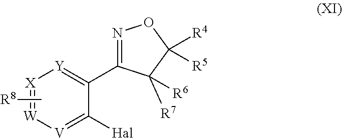

in which the radicals V, W, X, Y, R⁴, R⁵, R⁶, R⁷, R⁸ and Hal have the general, preferred and especially preferred meanings already indicated further hereinabove.

In a fifth embodiment of the intermediates, further subject matter of the present invention are also compounds of the formula (VIII)

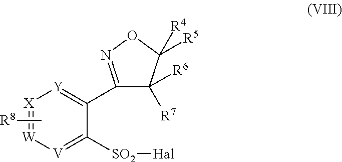

in which the radicals V, W, X, Y, R⁴, R⁵, R⁶, R⁷, R⁸ and Hal have the general, preferred and especially preferred meanings already indicated further hereinabove.

In a sixth embodiment of the intermediates, further subject matter of the present invention are also compounds of the formula (VI)

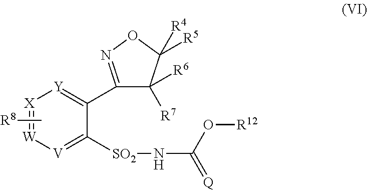

in which the radicals V, W, X, Y, R⁴, R⁵, R⁶, R⁷, R⁸, R¹² and Q have the general, preferred and especially preferred meanings already indicated further hereinabove.

Libraries of compounds of the formula (I) and/or their salts which can be synthesized in accordance with the abovementioned reactions can also be prepared in a parallelized fashion, which can be carried out manually or in a partially automated or fully automated fashion. Here, it is possible for example to automate the procedure of the reaction, the work-up or the purification of the products or intermediates.

Overall, this is understood as meaning a procedure as is described, for example, by D. Tiebes in Combinatorial Chemistry—Synthesis, Analysis, Screening (Editor Günther Jung), Verlag Wiley 1999, on pages 1 to 34.

To carry out the reaction and the work-up in a parallelized fashion, it is possible to use a series of apparatuses which are commercially available, for example Calypso reaction blocks from Barnstead International, Dubuque, Iowa 52004-0797, USA or reaction stations from Radleys, Shirehill, Saffron Walden, Essex, CB 11 3AZ, UK, or MuItiPROBE Automated Workstations from Perkin Elmar, Waltham, Mass. 02451, USA. To carry out a parallelized purification of compounds of the formula (I) and their salts, or the intermediates which are generated during the preparation, chromatographic apparatuses are available, inter alia, for example those from ISCO, Inc., 4700 Superior Street, Lincoln, Nebr. 68504, USA.

The apparatuses which have been listed lead to a modular procedure in which the individual passes are automated, but in which manual operations have to be carried out between the passes. This can be circumvented by employing partially or fully integrated automation systems where the respective automation modules are operated by, for example, robots. Said automation systems can be obtained commercially for example from Caliper, Hopkinton, Mass. 01748, USA.

Carrying out individual or a plurality of synthesis steps can be supported by using polymer-supported reagents/scavenger resins. A series of experimental protocols are described in the specialist literature, for example in ChemFiles, Vol. 4, No. 1, Polymer-Supported Scavengers and Reagents for Solution-Phase Synthesis (Sigma-Aldrich).

Compounds of the formula (I) and their salts can be prepared not only as in the methods described herein, but also fully or partially by solid-phase-supported methods. For this purpose, individual intermediates or all intermediates of the synthesis or of a synthesis adapted to suit the respective procedure are bound to a synthetic resin. Solid-phase-supported synthetic methods are described widely in the specialist literature, for example Barry A. Bunin in “The Combinatorial Index”, Academic Press, 1998 and Combinatorial Chemistry—Synthesis, Analysis, Screening (Editor Günther Jung), Verlag Wiley, 1999. The use of solid-phase-supported synthetic methods permits a series of protocols known from the literature, which, in turn, can be carried out manually or in an automated fashion. For example, the “teabag method” (Houghten, U.S. Pat. No. 4,631,211; Houghten et al., Proc. Natl. Acad. Sci., 1985, 82, 5131-5135) can be partly automated by products from IRORI, 11149 North Torrey Pines Road, La Jolla, Calif. 92037, USA. Solid-phase-supported parallel synthesis is successfully automated for example by equipment from Argonaut Technologies, Inc., 887 Industrial Road, San Carlos, Calif. 94070, USA or MultiSynTech GmbH, Wullener Feld 4, 58454 Witten, Germany. The reactions can, for example, also be carried out by means of IRORI technology in microreactors from Nexus Biosystems, 12140 Community Road, Poway, Calif. 92064, USA.

Both in the solid phase and in the liquid phase, carrying out individual, or a plurality of, synthesis steps can be supported by using microwave technology. A series of experimental protocols are described in the specialist literature, for example in Microwaves in Organic and Medicinal Chemistry (Editors C. O. Kappe and A. Stadler), published by Wiley, 2005.

The preparation according to the processes described herein yields compounds of the formula (I) and their salts in the form of substance collections, which are referred to as libraries. Subject matter of the present invention are also libraries which comprise at least two compounds of the formula (I) and their salts.

Another subject matter of the invention owing to the herbicidal property of the compounds of the formula (I) is also the use of the compounds of the formula (I) according to the invention as herbicides for controlling harmful plants.

Another subject matter of the invention owing to the herbicidal property of the compounds of the formula (I) is also the use of the compounds of the formula (I) according to the invention as herbicides for controlling harmful plants.

The application rate required of the compounds of the formula (I) varies as a function of the external conditions such as, inter alia, temperature, humidity and the nature of the herbicide used. It may vary within wide ranges, for example between 0.001 and 10 000 g/ha or more of active substance; preferably, however, it is between 0.5 and 5000 g/ha, by preference between 0.5 and 1000 g/ha and very especially preferably between 0.5 and 500 g/ha.

The compounds of the formula (I) according to the invention and their salts, hereinbelow synonymously together also referred to as compounds of the formula (I), have an outstanding herbicidal activity against a broad spectrum of economically important monocotyledonous and dicotyledonous harmful plants. The active substances also have a good effect on perennial harmful plants which produce shoots from rhizomes, rootstocks or other perennial organs and which are difficult to control. In this context, it is immaterial as to whether the substances are applied by the pre-plant method, the pre-emergence method or the post-emergence method.

By way of example, some representatives of the monocotyledonous and dicotyledonous weed flora which can be controlled by the compounds of the formula (I) according to the invention may be mentioned individually, without the enumeration being intended as a limitation to certain species.

Those where a good effect is obtained are, among the monocotyledonous weed species, for example Agrostis, Alopecurus, Apera, Avena, Brachicaria, Bromus, Dactyloctenium, Digitaria, Echinochloa, Eleocharis, Eleusine, Festuca, Fimbristylis, Ischaemum, Lolium, Monochoria, Panicum, Paspalum, Phalaris, Phleum, Poa, Sagittaria, Scirpus, Setaria, Sphenoclea, and Cyperus species predominantly from the annual group and, among the perennial species, Agropyron, Cynodon, Imperata and Sorghum, and also perennial Cyperus species.

Among the dicotyledonous weed species, the spectrum of action extends to species such as, for example, Galium, Viola, Veronica, Lamium, Stellaria, Amaranthus, Sinapis, Ipomoea, Matricaria, Abutilon and Sida among the annuals, and Convolvulus, Cirsium, Rumex and Artemisia among the perennial weeds. Furthermore, herbicidal activity is observed in dicotyledonous weeds such as Ambrosia, Anthemis, Carduus, Centaurea, Chenopodium, Cirsium, Convolvulus, Datura, Emex, Galeopsis, Galinsoga, Lepidium, Lindernia, Papaver, Portlaca, Polygonum, Ranunculus, Rorippa, Rotala, Seneceio, Sesbania, Solanum, Sonchus, Taraxacum, Trifolium, Urtica and Xanthium.

If the compounds according to the invention are applied to the surface before germination, either the emergence of the weed seedlings is prevented completely, or the weeds grow until they have reached the cotyledon stage, but then their growth is arrested and, ultimately, they die completely after three to four weeks have elapsed.

When, in the post-emergence method, the active substances of the formula (I) are applied to the green plant parts, the growth is arrested drastically and likewise very quickly after the treatment, and the weeds remain at the growth stage prevailing at the point in time of application or else die completely after a certain period of time has elapsed, so that, in this manner, competition by weeds, which is harmful to the crop plants, is eliminated at an early point in time and in a sustainable manner.

Although the compounds of the formula (I) according to the invention have an outstanding herbicidal activity against monocotyledonous and dicotyledonous weeds, crop plants of economically important crops such as, for example, wheat, barley, rye, rice, maize, sugar beet, cotton, oilseed rape and soya are damaged to a negligible extent only, or not at all. The present compounds are therefore highly suitable for the selective control of undesired plant growth in stands of agriculturally useful plants.

Moreover, the substances of the formula (I) according to the invention have outstanding growth-regulatory properties in crop plants. They engage in the plants' metabolism in the regulating fashion and can therefore be employed for selectively affecting plant constituents and for facilitating harvesting, such as, for example, by triggering desiccation and stunted growth. Moreover, they are also suitable for the general control and inhibition of undesired vegetative growth without destroying the plants in the process. The inhibition of vegetative growth plays an important role in many monocotyledonous and dicotyledonous crops since storage can thereby be reduced or prevented completely.

Due to the herbicidal and plant-growth-regulatory properties, the active substances can also be employed for controlling harmful plants in crops of plants which have been modified by genetic engineering and which are known or have yet to be developed. As a rule, the transgenic plants are distinguished by particularly advantageous properties, for example by resistances to certain pesticides, mainly certain herbicides, resistances to plant diseases or to causative organisms of plant diseases such as certain insects or microorganisms such as fungi, bacteria or viruses. Other particular properties relate for example to the harvested crop in respect of quantity, quality, solubility, composition and specific constituents. Thus, transgenic plants with an increased starch content or a modified starch quality or those with a different fatty acid composition of the harvested crop are known. Other particular properties may lie in a tolerance or resistance to abiotic stress factors, for example heat, cold, dryness, salt and ultraviolet radiation.

Preferred is the use of the compounds of the formula (I) or their salts according to the invention in economically important transgenic crops of useful plants and ornamentals, for example of cereals such as wheat, barley, rye, oats, millet/sorghum, rice, cassava and maize, or else crops of sugar beet, cotton, soya, oilseed rape, potato, tomato, pea and other vegetables.

Preferably, the compounds of the formula (I) can be employed as herbicides in crops of useful plants which are resistant, or have been made resistant by recombinant means, to the phytotoxic effects of the herbicides.

Traditional ways for generating novel plants which, in comparison with existing plants, have modified properties consist for example in classical breeding methods and the generation of mutants. Alternatively, it is possible to generate novel plants with modified properties with the aid of recombinant methods (see, for example, EP 0221044, EP 0131624). For example, the following have been described in several cases:
recombinant modifications of crop plants for modifying the starch which is synthesized in the plants (for example WO 92/011376, WO 92/014827, WO 91/019806),
transgenic crop plants which are resistant to certain herbicides of the glufosinate type (cf., for example, EP 0242236, EP 0242246) or of the glyphosate type (WO 92/000377) or of the sulfonylurea type (EP 0257993, U.S. Pat. No. 5,013,659),
transgenic crop plants, for example cotton, with the ability of producing Bacillus thuringiensis toxins (Bt toxins), which make the plants resistant to certain pests (EP 0142924, EP 0193259),
transgenic crop plants with a modified fatty acid composition (WO 91/013972),
recombinantly modified crop plants with novel constituents or secondary metabolites, for example novel phytoalexins, which bring about increased disease resistance (EP 0309862, EP 0464461),
recombinantly modified plants with reduced photorespiration which feature higher yields and higher stress tolerance (EP 0305398),
transgenic crop plants which produce pharmaceutically or diagnostically important proteins (“molecular pharming”),
transgenic crop plants which are distinguished by higher yields or better quality,
transgenic crop plants which are distinguished by a combination for example of the abovementioned novel properties (“gene stacking”).

A large number of molecular-biological techniques with the aid of which novel transgenic plants with modified properties can be generated are known in principle, see, for example, I. Potrykus and G. Spangenberg (eds.) Gene Transfer to Plants, Springer Lab Manual (1995), Springer Verlag Berlin, Heidelberg or Christou, “Trends in Plant Science” 1 (1996) 423-431).

To carry out such recombinant manipulations, it is possible to introduce, into plasmids, nucleic acid molecules which permit a mutagenesis or a sequence modification by recombining DNA sequences. With the aid of standard methods, for example, it is possible to carry out base substitutions, to remove part-sequences or to add natural or synthetic sequences. To link the DNA fragments to each other, it is possible to add adapters or linkers to the fragments, see, for example, Sambrook et al., 1989, Molecular Cloning, A Laboratory Manual, 2nd Ed., Cold Spring Harbor Laboratory Press, Cold Spring Harbor, N.Y.; or Winnacker “Gene and Klone” [genes and clones], VCH Weinheim 2nd Ed. 1996.

The generation of plant cells with a reduced activity of a gene product can be achieved for example by expression of at least one corresponding antisense RNA, a sense RNA for obtaining a cosuppression effect or the expression of at least one suitably constructed ribozyme which specifically cleaves transcripts of the abovementioned gene product.

Here, it is possible firstly to use DNA molecules which comprise the entire coding sequence of a gene product including any flanking sequences which may be present, or else DNA molecules which only comprise parts of the coding sequence, but these parts must be sufficiently long for bringing about an antisense effect in the cells. Another possibility is the use of DNA sequences which have a high degree of homology to the coding sequences of a gene product, but are not entirely identical.

When expressing nucleic acid molecules in plants, the protein synthetized can be localized in any compartment of the plant cell. To achieve localization in a particular compartment, however, it is possible for example to link the coding region to DNA sequences which ensure the localization in a particular compartment. Such sequences are known to the skilled worker (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). Expression of the nucleic acid molecules may also take place in the organelles of the plant cells.

The transgenic plant cells can be regenerated by known techniques to give intact plants. The transgenic plants may, in principle, take the form of plants of any plant species, i.e. both monocotyledonous and dicotyledonous plants.

Thus, it is possible to obtain transgenic plants which feature modified characteristics due to overexpression, suppression or inhibition of homologous (=natural) genes or gene sequences or by expressing heterologous (=foreign) genes or gene sequences.

The compounds of the formula (I) according to the invention can preferably be employed in transgenic crops which are resistant to growth substances, such as, for example, dicamba, or against herbicides which inhibit essential plant enzymes, for example acetolactate synthases (ALS), EPSP synthases, glutamine synthases (GS) or hydroxyphenylpyruvate dioxygenases (HPPD), or against herbicides from the group of the sulfonylureas, glyphosate, glufosinate or benzoylisoxazoles and analogous active substances, respectively.

When the active substances according to the invention are employed in transgenic crops, they show effects against harmful plants which can also be observed in other crops, but frequently also effects which are specific to the application in the respective transgenic crop, for example a modified or specifically widened weed spectrum which can be controlled, modified application rates which can be employed, preferably good combining ability with the herbicides to which the transgenic crop is resistant, and an effect on growth and yield of the transgenic crop plants.

The invention therefore also relates to the use of the compounds of the formula (I) according to the invention as herbicides for controlling harmful plants in transgenic crop plants.

The compounds according to the invention can be formulated in various ways, depending on the prevailing biological and/or chemical-physical parameters. The following are examples of possible formulations: wettable powders (WP), water-soluble powders (SP), water-soluble concentrates, emulsifiable concentrates (EC), emulsions (EW) such as oil-in-water and water-in-oil emulsions, sprayable solutions, suspension concentrates (SC), oil- or water-based dispersions, oil-miscible solutions, capsule suspensions (CS), dusts (DP), seed-dressing products, granules for broadcasting and soil application, granules (GR) in the form of microgranules, spray granules, absorption granules and adsorption granules, water-dispersible granules (WG), water-soluble granules (SG), ULV formulations, microcapsules and waxes.

These individual formulation types are known in principle and are described, for example, in: Winnacker-Küchler, “Chemische Technologie” [chemical technology], Volume 7, C. Hauser Verlag Munich, 4th Edition 1986; Wade van Valkenburg, “Pesticide Formulations”, Marcel Dekker, N.Y., 1973; K. Martens, “Spray Drying” Handbook, 3rd Ed. 1979, G. Goodwin Ltd. London.

The formulation auxiliaries required, such as inert materials, surfactants, solvents and further additives are likewise known and are described, for example, in: Watkins, “Handbook of Insecticide Dust Diluents and Carriers”, 2nd Ed., Darland Books, Caldwell N.J., H. v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [interface-active ethylene oxide adducts], Wiss. Verlagsgesell., Stuttgart 1976; Winnacker-Küchler, “Chemische Technologie”, Volume 7, C. Hauser Verlag Munich, 4th Edition 1986. Based on these formulations, it is also possible to prepare combinations with other pesticidally active substances such as, for example, insecticides, akaricides, herbicides, fungicides, and also with safeners, fertilizers and/or growth regulators, for example in the form of a ready mix or a tank mix.

Wettable powders are preparations which are uniformly dispersible in water and which, besides a diluent or inert substance, also comprise ionic and/or nonionic surfactants (wetting agents, dispersants) in addition to the active substance, for example polyoxyethylated alkylphenols, polyoxethylated fatty alcohols, polyoxethylated fatty amines, fatty alcohol polyglycol ether sulfates, alkanesulfonates, alkylbenzenesulfonates, sodium ligninsulfonate, sodium 2,2′-dinaphthylmethane-6,6′-disulfonate, sodium dibutylnaphthalenesulfonate or else sodium oleoylmethyltaurite. To prepare the wettable powders, the herbicidal active substances are ground finely, for example in customary apparatuses such as hammer mills, blower mills and air-jet mills, and simultaneously or subsequently mixed with the formulation auxiliaries.

Emulsifiable concentrates are prepared by dissolving the active substance in an organic solvent, for example butanol, cyclohexanone, dimethylformamide, xylene or else higher-boiling aromatics or hydrocarbons or mixtures of the organic solvents, with addition of one or more ionic and/or nonionic surfactants (emulsifiers). Examples of emulsifiers which can be used are: calcium salts of alkylarylsulfonic acids, such as calcium dodecylbenzenesulfonate, or nonionic emulsifiers such as fatty acid polyglycol esters, alkylarylpolyglycol ethers, fatty alcohol polyglycol ethers, propylene oxide/ethylene oxide condensates, alkyl polyethers, sorbitan esters such as, for example, sorbitan fatty acid esters or polyoxyethylene sorbitan esters such as, for example, polyoxyethylene sorbitan fatty acid esters.

Dusts are obtained by grinding the active substance with finely divided solid materials, for example talc, natural clays such as kaolin, bentonite and pyrophyllite, or diatomaceous earth.

Suspension concentrates may be water- or oil-based. They can be prepared for example by wet-grinding using commercially available bead mills and, if appropriate, an addition of surfactants as they have already been listed for example above in the case of the other formulation types.

Emulsions, for example oil-in-water emulsions (EW), can be prepared for example by means of stirrers, colloid mills and/or static mixers using aqueous-organic solvents and, if appropriate, surfactants as they have already been listed for example above in the case of the other formulation types.

Granules can be prepared either by spraying the active substance onto adsorptive granulated inert material or by applying active substance concentrates to the surface of carriers such as sand, kaolinites or granulated inert material by means of binders, for example polyvinyl alcohol, sodium polyacrylate or else mineral oils. Suitable active substances can also be granulated in the manner which is conventionally used for the preparation of fertilizer granules, if appropriate as a mixture with fertilizers.

As a rule, water-dispersible granules are prepared by the customary methods such as spray-drying, fluidized-bed granulation, disk granulation, mixing in high-speed mixers and extrusion without solid inert material. To prepare disk, fluidized-bed, extruder and spray granules, see, for example, the methods in “Spray-Drying Handbook” 3rd Ed. 1979, G. Goodwin Ltd., London; J. E. Browning, “Agglomeration”, Chemical and Engineering 1967, pages 147 et seq.; “Perry's Chemical Engineer's Handbook”, 5th Ed., McGraw-Hill, New York 1973, p. 8-57.

For further details on the formulation of plant protection products, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

As a rule, the agrochemical preparations comprise from 0.1 to 99% by weight, in particular from 0.1 to 95% by weight, of active substance of the formula (I).

In wettable powders, the active substance concentration amounts to for example approximately 10 to 90% by weight, the remainder to 100% is composed of conventional formulation components. In the case of emulsifiable concentrates, the active substance concentration may be approximately 1 to 90, preferably from 5 to 80% by weight. Formulations in the form of dust comprise from 1 to 30% by weight of active substance, preferably in most cases from 5 to 20% by weight of active substance, while sprayable solutions comprise from approximately 0.05 to 80, preferably from 2 to 50% by weight of active substance. In the case of water-dispersible granules, the active substance content depends partly on whether the active compound is present in liquid or solid form and on the granulation auxiliaries, fillers inter alia which are used. In the case of the water-dispersible granules, the active substance content is, for example, between 1 and 95% by weight, preferably between 10 and 80% by weight.

In addition, the abovementioned active substance formulation comprise, if appropriate, the adhesives, wetters, dispersants, emulsifiers, penetrants, preservatives, antifreeze agents, solvents, fillers, carriers, colorants, antifoam agents, evaporation inhibitors and agents which affect the pH and the viscosity which are customary in each case.

The compounds of the formula (I) or their salts can be employed as such or in the form of their preparations (formulations) as a combination with other pesticidally active substances such as, for example, insecticides, acaricides, nematicides, herbicides, fungicides, safeners, fertilizers and/or growth regulators, for example as a ready mix or as tank mixes. Combination partners which can be used for the active substances of the formula (I) according to the invention in mixed formulations or in a tank mix are, for example, known active substances which are based on the inhibition of, for example

acetolactate synthase, acetyl-coenzyme-A carboxylase, PS I, PS II, HPPDO, phytoene desaturase, protoporphyrinogen oxidase, glutamine synthetase, 5-enolpyruvylshikimate-3-phosphate synthetase or cellulose biosynthesis. Such compounds, and also other compounds which can be employed, whose mechanism of action is unknown to some degree or different, are described, for example, in Weed Research 26, 441-445 (1986), or “The Pesticide Manual”, 11th edition 1997 (hereinbelow also abbreviated to “PM”) and 12th edition 2000, The British Crop Protection Council and the Royal Soc. of Chemistry (publishers), and literature cited therein. Examples which may be mentioned of herbicides known from the literature which can be combined with the compounds of the formula (I) are the following active substances (note: the compounds are either referred to by the “common name” according to the International Organization for Standardization (ISO) or by the chemical name, if appropriate together with a customary code number):
acetochlor; acifluorfen(-sodium); aclonifen; AKH 7088, i.e. [[[1-[5-[2-chloro-4-(trifluoromethyl)phenoxy]-2-nitrophenyl]-2-methoxyethylidene]amino]oxy]acetic acid and its methyl ester; acrolein; alachlor; alloxydim (sodium); ametryn; amicarbazone, amidochlor, amidosulfuron; aminopyralid, amitrole; AMS, i.e. ammonium sulfamate; anilofos; asulam; atraton; atrazine; azafenidin, azimsulfuron (DPX-A8947); aziprotryn; barban; BAS 516 H, i.e. 5-fluoro-2-phenyl-4H-3,1-benzoxazin-4-one; BCPC; beflubutamid, benazolin (ethyl); benfluralin; benfuresate; bensulfuron (methyl); bensulide; bentazone; benzfendizone; benzobicyclon, benzofenap; benzofluor; benzoylprop (ethyl); benzthiazuron; bifenox; bialaphos; bifenox; bispyribac (sodium), borax; bromacil; bromobutide; bromofenoxim; bromoxynil; bromuron; buminafos; busoxinone; butachlor; butafenacil, butamifos; butenachlor; buthidazole; butralin; butroxydim, butylate; cacodylic acid; calcium chlorate; cafenstrole (CH-900); carbetamide; carfentrazone (ethyl); caloxydim, CDAA, i.e. 2-chloro-N,N-di-2-propenylacetamide; CDEC, i.e. 2-chloroallyl diethyldithiocarbamate; chlorflurenol (methyl); chlomethoxyfen; clethodim; clomeprop; chloramben; chlorazifop-butyl, chlormesulon; chlorbromuron; chlorbufam; chlorfenac; chlorflurecol-methyl; chloridazon; chlorimuron (ethyl); chloroacetic acid; chlornitrofen; chlorotoluron; chloroxuron; chlorpropham; chlorsulfuron; chlorthal (dimethyl); chlorthiamid; chlortoluron, cinidon (methyl and ethyl), cinmethylin; cinosulfuron; cisanilide; clefoxydim, clethodim; clodinafop and its ester derivatives (for example clodinafop-propargyl); clomazone; clomeprop; cloproxydim; clopyralid; clopyrasulfuron (methyl); cloransulam (methyl), cresole; cumyluron (JC 940); cyanamide; cyanazine; cycloate; cyclosulfamuron (AC 104); cycloxydim; cycluron; cyhalofop and its ester derivatives (for example butyl ester, DEH-112); cyperquat; cyprazine; cyprazole; daimuron; 2,4-D, 2,4-DB, 3,4-DA, 3,4-DB, 2,4-DEB, dalapon; dazomed; desmedipham; desmetryn; di-allate; dicamba; dichlobenil; ortho-dichlorobenzene; para-dichlorobenzene; dichlorprop; dichlorprop-P; diclofop and its esters such as diclofop-methyl; diclosulam, diethatyl (ethyl); difenoxuron; difenzoquat; difenzoquat-methylsulphate; diflufenican; diflufenzopyr, dimefuron; dimepiperate, dimethachlor; dimethametryn; dimethenamid (SAN-582H); dimethenamid-P; dimethazone, dimexyflam, dimethipin; diemethylarsinic acid; dinitramine; dinoseb; dinoterb; diphenamid; dipropetryn; diquat; diquat dibromide; dithiopyr; diuron; DNOC; 3,4-DP; DSMA; EBEP; eglinazine-ethyl; EL77, i.e. 5-cyano-1-(1,1-dimethylethyl)-N-methyl-1H-pyrazole-4-carboxamide; endothal; epoprodan, EPTC; esprocarb; ethalfluralin; ethametsulfuron (methyl); ethidimuron; ethiozin; ethofumesate; ethoxyfen and its esters (for example ethyl ester, HN-252); ethoxysulfuron, etobenzanid (HW 52); F5231, i.e. N-[2-chloro-4-fluoro-5-[4-(3-fluoropropyl)-4,5-dihydro-5-oxo-1H-tetrazol-1-yl]phenyl]-ethanesulfonamide; fenoprop; fenoxan, fenoxaprop and fenoxaprop-P and their esters, for example fenoxaprop-P-ethyl and fenoxaprop-ethyl; fenoxydim; fentrazamide, fenuron; ferrous sulfate; flamprop (methyl or isopropyl or isopropyl-L); flazasulfuron; floazulate, florasulam, fluazifop and fluazifop-P and their esters, for example fluazifop-butyl and fluazifop-P-butyl; fluazolate; flucarbazone (sodium), flucetosulfuron; fluchloralin; flufenacet; flufenpyr (ethyl); flumetsulam; flumeturon; flumiclorac (pentyl), flumioxazin (S-482); flumipropyn; fluometuron, fluorochloridone, fluorodifen; fluoroglycofen (ethyl); flupoxam (KNW-739); flupropacil (UBIC-4243); flupropanate, flupyrsulfuron (methyl or sodium), flurenol (butyl), fluridone; fluorochloridone; fluoroxypyr (meptyl); flurprimidol; flurtamone; fluthiacet (methyl) (KIH-9201); fluthiamide; fomesafen; foramsulfuron; fosamine; furyloxyfen; glufosinate (ammonium); glyphosate (isopropylammonium); halosafen; halosulfuron (methyl) and its esters (for example methyl ester, NC-319); haloxyfop and its esters; haloxyfop-P (=R-haloxyfop) and its esters; HC-252; hexazinone; imazamethabenz (methyl); imazapyr; imazaquin and salts such as the ammonium salt; imazamethapyr, imazamox, imazapic, imazethamethapyr; imazethapyr; imazosulfuron; indanofan, iodomethane; iodosulfuron (methylsodium); ioxynil; isocarbamid; isopropalin; isoproturon; isouron; isoxaben; isoxachlortole, isoxaflutole, isoxapyrifop; karbutilate; lactofen; lenacil; linuron; MAA; MAMA; MCPA; MCPA-2-ethylhexyl; MCPA-thioethyl; MCPB; mecoprop; mecoprop-P; mefenacet; mefluidid; mesosulfuron (methyl); mesotrione, metamifop; metamitron; metazachlor; methabenzthiazuron; metham; methazole; methoxyphenone; methylarsonic acid; methyldymron; methyl isothiocyanate; metabenzuron, metamifop; methobenzuron; metobromuron; (alpha-)metolachlor; S-metolachlor; metosulam (XRD 511); metoxuron; metribuzin; metsulfuron-methyl; MK-616; MH; molinate; monalide; monocarbamide dihydrogensulfate; monolinuron; monuron; monosulfuron; MSMA; MT 128, i.e. 6-chloro-N-(3-chloro-2-propenyl)-5-methyl-N-phenyl-3-pyridazinamine; MT 5950, i.e. N-[3-chloro-4-(1-methylethyl)phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclophen; nitralin; nitrofen; nitrofluorfen; nonanoic acid; norflurazon; oleic acid (fatty acid); orbencarb; orthosulfamuron; oryzalin; oxadiargyl (RP-020630); oxadiazon; oxasulfuron, oxaziclomefone, oxyfluorfen; paraquat; paraquat dichloide; pebulate; pelargonic acid, pendimethalin; penoxsulam; pentachlorophenol; pentanochlor; pentoxazone, perfluidone; phenisopham; phenmedipham (ethyl); pethoxamid; picloram; picolinafen, pinoxaden, piperophos; piributicarb; pirifenopbutyl; pretilachlor; primisulfuron (methyl); potassium arsenite; potassium azide; procarbazone (sodium), procyazine; prodiamine; profluazol; profluralin; profoxydim; proglinazine (ethyl); prometon; prometryn; propachlor; propanil; propaquizafop and its esters; propazine; propham; propisochlor; propoxycarbazone (sodium) (BAY MKH 6561); propyzamide; prosulfalin; prosulfocarb; prosulfuron (CGA-152005); prynachlor; pyraclonil; pyraflufen (ethyl), pyrasulfotole; pyrazolinate; pyrazon; pyrazosulfuron (ethyl); pyrazoxyfen; pyribambenz-isopropyl; pyribenzoxim, pyributicarb, pyridafol, pyridate; pyriftalide; pyrimidobac (methyl), pyrimisulfan, pyrithiobac (sodium) (KIH-2031); pyroxasulfone; pyroxofop and its esters (for example propargyl ester); pyroxsulam (triflosulam); quinclorac; quinmerac; quinoclamine, quinofop and its ester derivatives, quizalofop and quizalofop-P and their ester derivatives, for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]-4,5,6,7-tetrahydro-2H-indazole; saflufenacil (CAS-RN: 372137-35-4); secbumeton; sethoxydim; siduron; simazine; simetryn; SN 106279, i.e. 2-[[7-[2-chloro-4-(trifluoromethyl)phenoxy]-2-naphthalenyl]-oxy]propanoic acid and its methyl ester; SMA; sodium arsenite; sodium azide; sodium chlorate; sulcotrione, sulfentrazon (FMC-97285, F-6285); sulfazuron; sulfometuron (methyl); sulfosate (ICI-A0224); sulfosulfuron, 2,3,6-TBA; TCA (sodium); tebutam (GCP-5544); tebuthiuron; tefuryltrione, tembotrione, tepraloxydim, terbacil; terbucarb; terbuchlor; terbumeton; terbuthylazine; terbutryn; TFH 450, i.e. N,N-diethyl-3-[(2-ethyl-6-methylphenyl)sulfonyl]-1H-1,2,4-triazol-1-carboxamide; thenylchlor (NSK-850); thiafluamide, thiazafluoron; thiazopyr (Mon-13200); thidiazimin (SN-24085); thiencarbazone-methyl, thifensulfuron (methyl); thiobencarb; tiocarbazil; tralkoxydim; tri-allate; triasulfuron; triaziflam, triazofenamide; tribenuron (methyl); tricamba; triclopyr; tridiphane; trietazine; trifloxysulfuron (sodium); trifluralin; triflusulfuron-methyl and esters (for example methyl ester, DPX-66037); trihydroxytriazine; trimeturon; tritosulfuron; tropramezone; tsitodef; vernolate; [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester; WL 110547, i.e. 5-phenoxy-1-[3-(trifluoromethyl)phenyl]-1H-tetrazole; UBH-509; D-489; LS 82-556, i.e. [(S)-3-N-(methylbenzyl)carbamoyl-5-propionyl-2,6-lutidine]; KPP-300; NC-324; NC-330; KH-218; DPX-N8189; SC-0774; DOWCO-535; DK-8910; V-53482; PP-600; MBH-001; ET-751, i.e. ethyl [2-chloro-5-(4-chloro-5-difluoromethoxy-1-methyl-1H-pyrazol-3-yl)-4-fluorophenoxy]acetate; KIH-6127, i.e. pyriminobac-methyl; KIH-2023, i.e. bispyribac-sodium; and SYP-249, i.e. ethyl 2-{2-nitro-5-[(2-chloro-4-trifluoromethyl)phenoxy]benzoxy}-3-methyl-3-butenoate; SYN-523.

The selective control of harmful plants in crops of useful plants and ornamentals is of particular interest. Although the compounds of the formula (I) according to the invention already display very good to sufficient selectivity in many crops, phytotoxicity symptoms on the crop plants may occur, in principle, in some crops and especially also in the case of mixtures with other herbicides which are less selective. In this respect, combinations of compounds of the formula (I) according to the invention which are of particular interest are those which comprise the compounds of the formula (I) or their combinations with other herbicides or pesticides and safeners. The safeners, which are employed in an antidote-effective content, reduce the phytotoxic side-effects of the herbicides/pesticides employed, for example economically important crops such as cereals (wheat, barley, rye, maize, rice, millet/sorghum), sugar beet, sugar cane, oilseed rape, cotton and soya beans, preferably cereals. The following groups of compounds are examples which are suitable as safeners for the compounds (I), alone or else in their combinations with further pesticides:

The safeners are preferably selected from the group consisting of:
S1) Compounds of the Formula (S1)

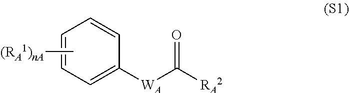

where the symbols and indices have the following meanings:
n_{A} is a natural number from 0 to 5, preferably from 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic radical from the group consisting of partially unsaturated or aromatic five-membered heterocycles having 1 to 3 heteroring atoms of the group N and O, where at least one nitrogen atom and at most one oxygen atom is present in the ring, preferably a radical from the group consisting of (W_{A}¹) to (W_{A}⁴),

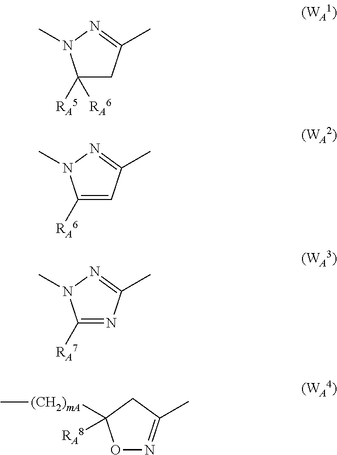

m_{A} is 0 or 1;
R_{A}² is OR_{A}³, SR_{A}³ or NR_{A}³R_{A}⁴ or a saturated or unsaturated 3- to 7-membered heterocycle having at least one nitrogen atom and up to 3 heteroatoms, preferably from the group consisting of O and S, which is attached via the nitrogen atom to the carbonyl group in (S1) and which is unsubstituted or substituted by radicals from the group consisting of (C₁-C₄)alkyl, (C₁-C₄)alkoxy and optionally substituted phenyl, preferably a radical of the formula OR_{A}³, NHR_{A}⁴ or N(CH₃)₂, in particular of the formula OR_{A}³;
R_{A}³ is hydrogen or an unsubstituted or substituted aliphatic hydrocarbon radical having preferably a total of 1 to 18 carbon atoms;
R_{A}⁴ is hydrogen, (C₁-C₆)alkyl, (C₁-C₆)alkoxy or substituted or unsubstituted phenyl;
R_{A}⁵ is H, (C₁-C₈)alkyl, (C₁-C₈)haloalkyl, (C₁-C₄)alkoxy-(C₁-C₈)alkyl, cyano or COOR_{A}⁹ where R_{A}⁹ is hydrogen, (C₁-C₈)alkyl, (C₁-C₈)haloalkyl, (C₁-C₄)alkoxy-(C₁-C₄)alkyl, (C₁-C₆)hydroxyalkyl, (C₃-C₁₂)cycloalkyl or tri-(C₁-C₄)alkylsilyl;
R_{A}⁶, R_{A}⁷, R_{A}⁸ are identical or different and are hydrogen, (C₁-C₈)alkyl, (C₁-C₈)haloalkyl, (C₃-C₁₂)cycloalkyl or substituted or unsubstituted phenyl;
   preferably:
a) compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds, as described in WO-A-91/07874;
b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4), and related compounds, as described in EP-A-333 131 and EP-A-269 806;
c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds, as described, for example, in EP-A-268554;
d) compounds of the triazolecarboxylic acid type, preferably compounds such as fenchlorazole(-ethyl), i.e. ethyl 1-(2,4-dichlorophenyl)-5-trichloro-methyl-(1H)-1,2,4-triazole-3-carboxylate (S1-7), and related compounds, as described in EP-A-174 562 and EP-A-346 620;
e) compounds of the 5-benzyl- or 5-phenyl-2-isoxazoline-3-carboxylic acid or the 5,5-diphenyl-2-isoxazoline-3-carboxylic acid (S1^{e}) type, preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds, as described in WO-A-91/08202, or 5,5-diphenyl-2-isoxazolinecarboxylic acid (S1-10) or ethyl 5,5-diphenyl-2-isoxazolinecarboxylate (S1-11) (“isoxadifenethyl”) or n-propyl 5,5-diphenyl-2-isoxazolinecarboxylate (S1-12) or ethyl 5-(4-fluorophenyl)-5-phenyl-2-isoxazoline-3-carboxylate (S1-13), as described in the patent application WO-A-95/07897.
S2) Quinoline Derivatives of the Formula (S2)

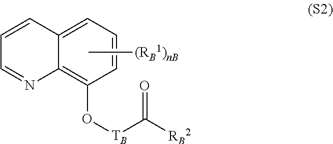

where the symbols and indices have the following meanings:
R_{B}¹ is halogen, (C₁-C₄)alkyl, (C₁-C₄)alkoxy, nitro or (C₁-C₄)haloalkyl;
n_{B} is a natural number from 0 to 5, preferably from 0 to 3;
R_{B}² is OR_{B}³, SR_{B}³ or NR_{B}³R_{B}⁴ or a saturated or unsaturated 3- to 7-membered heterocycle having at least one nitrogen atom and up to 3 heteroatoms, preferably from the group consisting of O and S, which is attached via the nitrogen atom to the carbonyl group in (S2) and is unsubstituted or substituted by radicals from the group consisting of (C₁-C₄)alkyl, (C₁-C₄)alkoxy or optionally substituted phenyl, preferably a radical of the formula OR_{B}³, NHR_{B}⁴ or N(CH₃)₂, in particular of the formula OR_{B}³;
R_{B}³ is hydrogen or an unsubstituted or substituted aliphatic hydrocarbon radical having preferably a total of 1 to 18 carbon atoms;
R_{B}⁴ is hydrogen, (C₁-C₆)alkyl, (C₁-C₆)alkoxy or substituted or unsubstituted phenyl;

T_{B} is a (C₁- or C₂)alkanediyl chain which is unsubstituted or substituted by one or two (C₁-C₄)alkyl radicals or by [(C₁-C₃)alkoxy]carbonyl;

preferably:
a) compounds of the 8-quinolinoxyacetic acid (S2^{a}) type, preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5), methyl (5-chloro-8-quinolinoxy)acetate (S2-6), allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and (5-chloro-8-quinolinoxy)acetic acid (S2-10), its hydrates and salts, for example its lithium, sodium, potassium, calcium, magnesium, aluminum, iron, ammonium, quarternary ammonium, sulfonium or phosphonium salts, as described in WO-A-2002/34048;
b) compounds of the (5-chloro-8-quinolinoxy)malonic acid (S2^{b}) type, preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Compounds of the Formula (S3)

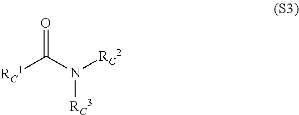

where the symbols and indices have the following meanings:
R_{C}¹ is (C₁-C₄)alkyl, (C₁-C₄)haloalkyl, (C₂-C₄)alkenyl, (C₂-C₄)haloalkenyl, (C₃-C₇)cycloalkyl, preferably dichloromethyl;
R_{C}², R_{C}³ are identical or different and are hydrogen, (C₁-C₄)alkyl, (C₂-C₄)alkenyl, (C₂-C₄)alkynyl, (C₁-C₄)haloalkyl, (C₂-C₄)haloalkenyl, (C₁-C₄)alkylcarbamoyl-(C₁-C₄)alkyl, (C₂-C₄)alkenylcarbamoyl-(C₁-C₄)alkyl, (C₁-C₄)alkoxy-(C₁-C₄)alkyl, dioxolanyl-(C₁-C₄)alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring;
   preferably
   active substances of the dichloroacetamide type which are frequently used as pre-emergence safeners (soil-acting safeners), such as, for example,
   “dichlormid” (=N,N-diallyl-2,2-dichloroacetamide) (S3-1),
   “R-29148” (=3-dichloroacetyl-2,2,5-trimethyl-1,3-oxazolidine) from Stauffer (S3-2),
   “R-28725” (=3-dichloroacetyl-2,2-dimethyl-1,3-oxazolidine) from Stauffer (S3-3),
   “benoxacor” (=4-dichloroacetyl-3,4-dihydro-3-methyl-2H-1,4-benzoxazine) (S3-4),
   “PPG-1292” (=N-allyl-N-[(1,3-dioxolan-2-yl)methyl]dichloroacetamide) from PPG Industries (S3-5),
   “DKA-24” (=N-allyl-N-[(allylaminocarbonyl)methyl]dichloroacetamide) from Sagro-Chem (S3-6),
   “AD-67” or “MON 4660” (=3-dichloroacetyl-1-oxa-3-azaspiro[4,5]decane) from Nitrokemia or Monsanto (S3-7),
   “TI-35” (1-dichloroacetylazepane) from TRI-Chemical RT (S3-8),
   “diclonon” (dicyclonone) or “BAS145138” or “LAB145138” (S3-9) (3-dichloroacetyl-2,5,5-trimethyl-1,3-diazabicyclo[4.3.0]nonane) from BASF,
   “furilazole” or “MON 13900” ((RS)-3-dichloroacetyl-5-(2-furyl)-2,2-dimethyl-oxazolidine) (S3-10) and its (R) isomer (S3-11).
S4) N-Acylsulfonamides of the Formula (S4) and Their Salts

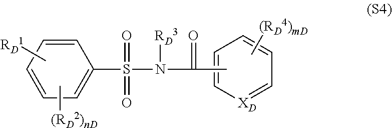

in which the symbols and indices have the following meanings
X_{D} is CH or N;
R_{D}¹ is CO—NR_{D}⁵R_{D}⁶ or NHCO—R_{D}⁷;
R_{D}² is halogen, (C₁-C₄)haloalkyl, (C₁-C₄)haloalkoxy, nitro, (C₁-C₄)alkyl, (C₁-C₄)alkoxy, (C₁-C₄)alkylsulfonyl, (C₁-C₄)alkoxycarbonyl or (C₁-C₄)alkylcarbonyl;
R_{D}³ is hydrogen, (C₁-C₄)alkyl, (C₂-C₄)alkenyl or (C₂-C₄)alkynyl;
R_{D}⁴ is halogen, nitro, (C₁-C₄)alkyl, (C₁-C₄)haloalkyl, (C₁-C₄)haloalkoxy, (C₃-C₆)cycloalkyl, phenyl, (C₁-C₄)alkoxy, cyano, (C₁-C₄)alkylthio, (C₁-C₄)alkylsulfinyl, (C₁-C₄)alkylsulfonyl, (C₁-C₄)alkoxycarbonyl or (C₁-C₄)alkylcarbonyl;
R_{D}⁵ is hydrogen, (C₁-C₆)alkyl, (C₃-C₆)cycloalkyl, (C₂-C₆)alkenyl, (C₂-C₆)alkynyl, (C₅-C₆)cycloalkenyl, phenyl or 3- to 6-membered heterocyclyl containing v_{D} heteroatoms from the group consisting of nitrogen, oxygen and sulfur, where the seven last mentioned radicals are substituted by v_{D} substituents from the group consisting of halogen, (C₁-C₆)alkoxy, (C₁-C₆)haloalkoxy, (C₁-C₂)alkylsulfinyl, (C₁-C₂)alkylsulfonyl, (C₃-C₆)cycloalkyl, (C₁-C₄)alkoxycarbonyl, (C₁-C₄)alkylcarbonyl and phenyl and, in the case of cyclic radicals, also (C₁-C₄)alkyl and (C₁-C₄)haloalkyl;
R_{D}⁶ is hydrogen, (C₁-C₆)alkyl, (C₂-C₆)alkenyl or (C₂-C₆)alkynyl, where the three last mentioned radicals are substituted by v_{D} radicals from the group consisting of halogen, hydroxy, (C₁-C₄)alkyl, (C₁-C₄)alkoxy and (C₁-C₄)alkylthio, or
   R_{D}⁵ and R_{D}⁶ together with the nitrogen atom carrying them form a pyrrolidinyl or piperidinyl radical;
R_{D}⁷ is hydrogen, (C₁-C₄)alkylamino, di-(C₁-C₄)alkylamino, (C₁-C₆)alkyl, (C₃-C₆)cycloalkyl, where the 2 last mentioned radicals are substituted by v_{D} substituents from the group consisting of halogen, (C₁-C₄)alkoxy, halo(C₁-C₆)alkoxy and (C₁₋C₄)alkylthio and, in the case of cyclic radicals, also (C₁-C₄)alkyl and (C₁-C₄)haloalkyl;
n_{D} is 0, 1 or 2;
m_{D} is 1 or 2;
v_{D} is 0, 1, 2 or 3;
   from among these, preference is given to compounds of the N-acylsulfonamide type (S4^{a}), for example of the formula below, which are known, for example, from WO-A-97/45016

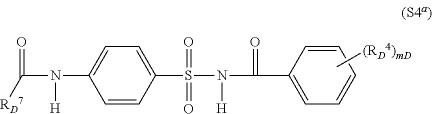

in which
R_{D}⁷ is (C₁-C₆)alkyl, (C₃-C₆)cycloalkyl, where the 2 last mentioned radicals are substituted by v_{D} substituents from the group consisting of halogen, (C₁-C₄)alkoxy, halo(C₁-C₆)alkoxy and (C₁-C₄)alkylthio and, in the case of cyclic radicals, also (C₁-C₄)alkyl and (C₁-C₄)haloalkyl;
R_{D}⁴ is halogen, (C₁-C₄)alkyl, (C₁-C₄)alkoxy, CF₃,
m_{D} is 1 or 2;
v_{D} is 0, 1, 2 or 3;
   and also
   acylsulfamoylbenzamides, for example of the formula (S4^{b}) below, which are known, for example, from WO-A-99/16744,

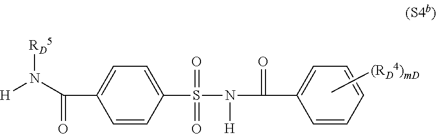

for example those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-Cl-2-OMe (S4-4) and
R_{D}⁵=isopropyl and (R_{D}⁴)=2-OMe (S4-5);
   and also
   compounds of the N-acylsulfamoylphenylurea type of the formula (S4^{c}), which are known, for example, from EP-A-365484 in which

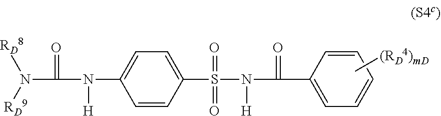

R_{D}⁸ and R_{D}⁹ independently of one another are hydrogen, (C₁-C₈)alkyl, (C₃-C₈)cycloalkyl, (C₃-C₆)alkenyl, (C₃-C₆)alkynyl,
R_{D}⁴ is halogen, (C₁-C₄)alkyl, (C₁-C₄)alkoxy, CF₃
m_{D} is 1 or 2;
   from among these in particular
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea.
S5) Active substances from the class of the hydroxyaromatics and aromatic-aliphatic carboxylic acid derivatives (S5), for example
   ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active substances from the class of the 1,2-dihydroquinoxalin-2-ones (S6), for example
   1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds of the formula (S7), as described in WO-A-1998/38856

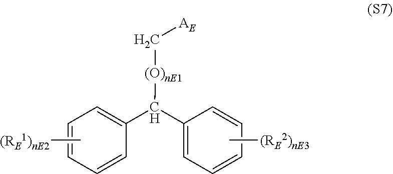

in which the symbols and indices have the following meanings:
R_{E}¹, R_{E}² independently of one another are halogen, (C₁-C₄)alkyl, (C₁-C₄)alkoxy, (C₁-C₄)haloalkyl, (C₁-C₄)alkylamino, di-(C₁-C₄)alkylamino, nitro;
A_{E} is COOR_{E}³ or COSR_{E}⁴
R_{E}³, R_{E}⁴ independently of one another are hydrogen, (C₁-C₄)alkyl, (C₂-C₆)alkenyl, (C₂-C₄)alkynyl, cyanoalkyl, (C₁-C₄)haloalkyl, phenyl, nitrophenyl, benzyl, halobenzyl, pyridinylalkyl and alkylammonium,
n_{E}¹ is 0 or 1
n_{E}², n_{E}³ independently of one another are 0, 1 or 2,
   preferably:
   diphenylmethoxy acetic acid,
   ethyl diphenylmethoxy acetate,
   methyl diphenylmethoxy acetate (CAS-Reg. No. 41858-19-9) (S7-1).
S8) Compounds of the formula (S8) as described in WO-A-98/27049

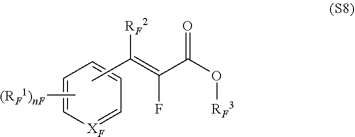

in which
X_{F} is CH or N,
n_{F} in the event that X_{F}═N, is an integer from 0 to 4 and in the event that X_{F}=CH, is an integer from 0 to 5,
R_{F}¹ is halogen, (C₁-C₄)alkyl, (C₁-C₄)haloalkyl, (C₁-C₄)alkoxy, (C₁-C₄)haloalkoxy, nitro, (C₁-C₄)alkylthio, (C₁-C₄)-alkylsulfonyl, (C₁-C₄)alkoxycarbonyl, optionally substituted phenyl, optionally substituted phenoxy,
R_{F}² is hydrogen or (C₁-C₄)alkyl,
R_{F}³ is hydrogen, (C₁-C₈)alkyl, (C₂-C₄)alkenyl, (C₂-C₄)alkynyl, or aryl, each of the abovementioned C-comprising radicals being unsubstituted or substituted by one or more, preferably up to three, identical or different radicals selected from the group consisting of halogen and alkoxy, or their salts,
   preferably compounds in which
X_{F} is CH,
n_{F} is an integer from 0 to 2,
R_{F}¹ is halogen, (C₁-C₄)alkyl, (C₁-C₄)haloalkyl, (C₁-C₄)alkoxy, (C₁-C₄)haloalkoxy,
R_{F}² is hydrogen or (C₁-C₄)alkyl,
R_{F}³ is hydrogen, (C₁-C₈)alkyl, (C₂-C₄)alkenyl, (C₂-C₄)alkynyl, or aryl, where each of the abovementioned C-comprising radicals is unsubstituted or substituted by one or more, preferably up to three, identical or different radicals selected from the group consisting of halogen and alkoxy, or their salts.
S9) Active substances from the class of the 3-(5-tetrazolylcarbonyl)-2-quinolones (S9), for example
   1,2-dihydro-4-hydroxy-1-ethyl-3-(5-tetrazolylcarbonyl)-2-quinolone (CAS-Reg. No. 219479-18-2), 1,2-dihydro-4-hydroxy-1-methyl-3-(5-tetrazolylcarbonyl)-2-quinolone (CAS-Reg. No. 95855-00-8), as described in WO-A-1999/000020.
S10) Compounds of the formulae (S10^{a}) or (S10^{b})
   as described in WO-A-2007/023719 and WO-A-2007/023764

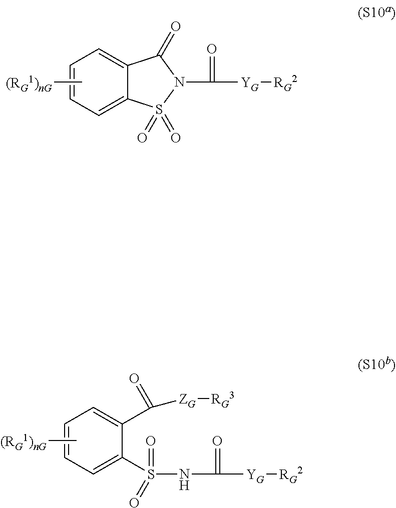

in which
R_{G}¹ is halogen, (C₁-C₄)alkyl, methoxy, nitro, cyano, CF₃, OCF₃
Y_{G}, Z_{G} independently of one another are O or S,
n_{G} is an integer from 0 to 4,
R_{G}² is (C₁-C₁₆)alkyl, (C₂-C₆)alkenyl, (C₃-C₆)cycloalkyl, aryl; benzyl, halobenzyl,
R_{G}³ is hydrogen or (C₁-C₆)alkyl.
S11) Active substances of the oxyimino compound type (S11), which are known as seed-dressing agents, such as, for example,
   “oxabetrinil” ((Z)-1,3-dioxolan-2-ylmethoxyimino(phenyl)acetonitrile) (S11-1), which is known as seed-dressing safener for millet/sorghum against metolachlor-induced damage,
   “fluxofenim” (1-(4-chlorophenyl)-2,2,2-trifluoro-1-ethanone O-(1,3-dioxolan-2-ylmethyl) oxime) (S11-2), which is known as seed-dressing safener for millet/sorghum against metolachlor-induced damage, and
   “cyometrinil” or “CGA-43089” ((Z)-cyanomethoxyimino(phenyl)acetonitrile) (S11-3), which is known as seed-dressing safener for millet/sorghum against metolachlor-induced damage.
S12) Active substances from the class of the isothiochromanones (S12), such as, for example, methyl [(3-oxo-1H-2-benzothiopyran-4(3H)-ylidene)methoxy]acetate (CAS-Reg. No. 205121-04-6) (S12-1) and related compounds from WO-A-1998/13361.
S13) One or more compounds selected from the group (S13) consisting of:
   “naphthalic anhydride” (1,8-naphthalenedicarboxylic anhydride) (S13-1), which is known as seed-dressing safener for maize against thiocarbamate-herbicide-induced damage,
   “fenclorim” (4,6-dichloro-2-phenylpyrimidine) (S13-2), which is known as safener for pretilachlor in seeded rice,
   “flurazole” (benzyl 2-chloro-4-trifluoromethyl-1,3-thiazole-5-carboxylate) (S13-3), which is known as seed-dressing safener for millet/sorghum against alachlor and metolachlor-induced damage,
   “CL 304415” (CAS-Reg. No. 31541-57-8) (4-carboxy-3,4-dihydro-2H-1-benzopyrane-4-acetic acid) (S13-4) from American Cyanamide, which is known as safener for maize against damage induced by imidazolinones,
   “MG 191” (CAS-Reg. No. 96420-72-3) (2-dichloromethyl-2-methyl-1,3-dioxolane) (S13-5) from Nitrokemia, which is known as safener for maize,
   “MG-838” (CAS-Reg. No. 133993-74-5) (2-propenyl 1-oxa-4-azaspiro[4.5]decane-4-carbodithioate) (S13-6) from Nitrokemia,
   “disulfoton” (O,O-diethyl S-2-ethylthioethyl phosphorodithioate) (S13-7),
   “dietholate” (O,O-diethyl O-phenyl phosphorothioate) (S13-8),
   “mephenate” (4-chlorophenyl methylcarbamate) (S13-9).
S14) Active substances which, in addition to a herbicidal action against harmful plants, also have safener action on crop plants such as rice, such as, for example,
   “dimepiperate” or “MY-93” (S-1-methyl-1-phenylethyl piperidine-1-carbothioate), which is known as safener for rice against damage induced by the herbicide molinate,
   “daimuron” or “SK 23” (1-(1-methyl-1-phenylethyl)-3-p-tolylurea), which is known as safener for rice against damage induced by the herbicide imazosulfuron,
   “cumyluron”=“JC-940” (3-(2-chlorophenylmethyl)-1-(1-methyl-1-phenylethyl)urea, see JP-A-60087254), which is known as safener for rice against damage induced by a number of herbicides,
   “methoxyphenone” or “NK 049” (3,3′-dimethyl-4-methoxybenzophenone), which is known as safener for rice against damage induced by a number of herbicides,
   “CSB” (1-bromo-4-(chloromethylsulfonyl)benzene) (CAS Reg. No. 54091-06-4 from Kumiai), which is known as safener against damage induced by a number of herbicides in rice.
S15) Active substances which are employed predominantly as herbicides, but which also have a safener action on crop plants, for example
(2,4-dichlorophenoxy)acetic acid (2,4-D),
(4-chlorophenoxy)acetic acid,
(R,S)-2-(4-chloro-o-tolyloxy)propionic acid (mecoprop),
4-(2,4-dichlorophenoxy)butyric acid (2,4-DB),
(4-chloro-o-tolyloxy)acetic acid (MCPA),
4-(4-chloro-o-tolyloxy)butyric acid,
4-(4-chlorophenoxy)butyric acid,
3,6-dichloro-2-methoxybenzoic acid (dicamba),
1-(ethoxycarbonyl)ethyl 3,6-dichloro-2-methoxybenzoate (lactidichloroethyl).

Preferred herbicide/safener combinations are those which comprise (A) a herbicidally active amount of one or more compounds of the formula (I) or their salts, and (B) an antidote-effective amount of one or more safeners.

For the purposes of the invention, a herbicidally active amount means an amount of one or more herbicides which is suitable for exerting a negative effect on the growth of plants. For the purposes of the invention, an antidote-effective amount means an amount of one or more safeners which is suitable for reducing the phytotoxic effect of plant protection actives (for example of herbicides) on crop plants.

Some of the safeners are already known as herbicides and thus exert not only a herbicidal effect on harmful plants, but also a protective effect on the crop plants.

The weight ratio of herbicide (mixture) to safener will generally depend on the application rate of herbicide and on the efficacy of the safener in question and can vary within wide limits, for example in the range of from 200:1 to 1:200, preferably 100:1 to 1:100, in particular 20:1 to 1:20. The safeners can be formulated together with further herbicides/pesticides analogously to the compounds of the formula (I) or their mixtures and provided, and used, as a ready mix or a tank mix together with the herbicides.

For use, the formulations, which are present in commercially available form, are, if appropriate, diluted in the customary manner, for example by means of water in the case of wettable powders, emulsifiable concentrates, dispersions and water-dispersible granules. Preparations in the form of dusts, soil granules or granules for broadcasting and sprayable solutions are usually not diluted any further with other inert substances prior to use.

The application rate required, for the compounds of the formula (I), varies as a function of the external conditions such as temperature, humidity, the nature of the herbicide used and others. It can vary within wide limits, for example between 0.001 and 10.0 kg/ha or more of active substance, but it is preferably between 0.005 and 5 kg/ha.

The present invention is illustrated in greater detail with reference to the examples which follow which, however, impose no limitation whatsoever on the invention.

### A. SYNTHESIS EXAMPLES

### 1. N-[(4-Chloro-6-methoxypyrimidin-2-yl)carbamoyl]-3-(4,5-dihydroisoxazol-3-yl)-pyridine-2-sulfonamide (Ia-1)

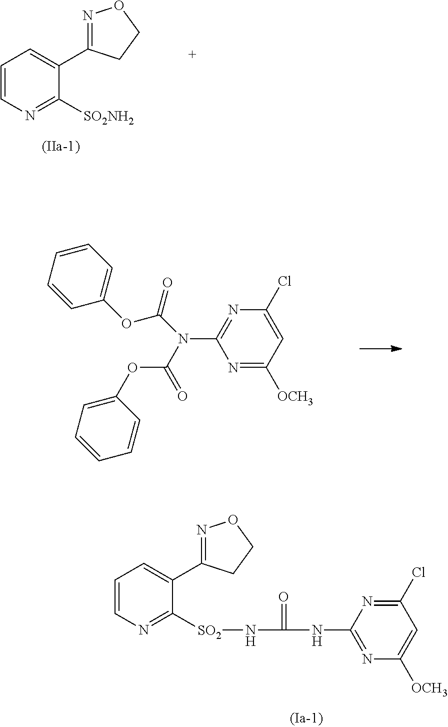

230 mg (1 mmol) of 3-(4,5-dihydroisoxazol-3-yl)pyridine-2-sulfonamide (IIa-1) are dissolved in 10 ml of acetonitrile and treated with 400 mg (1 mmol) of diphenyl (4-chloro-6-methoxypyrimidin-2-yl)imidodicarbonate (cf. WO 1996/022284). 160 mg (1 mmol) of DBU (diazabicycloundecene) are added dropwise with stirring. After the mixture has been left to stand for 1 hour at 20° C., it is poured into a mixture of methylene chloride and aqueous hydrochloric acid (5% strength). The organic phase is washed twice with water and once with saturated sodium chloride solution. After drying with magnesium sulfate, the organic phase is concentrated in vacuo. The residue is triturated in diethyl ether and filtered off with suction. The resulting crystals are stirred in isopropanol, filtered off with suction and dried. This gives 200 mg (0.45 mmol) of N-[(4-chloro-6-methoxypyrimidin-2-yl)carbamoyl]-3-(4,5-dihydroisoxazol-3-yl)pyridine-2-sulfonamide (Ia-1) of purity (HPLC) 92.8%.

The compounds of the formula (I) which are described in the tables hereinbelow are obtained according to, or analogously to, the above-described synthesis example:

TABLE 1a
Compounds of the formula (Ia)
(Ia)

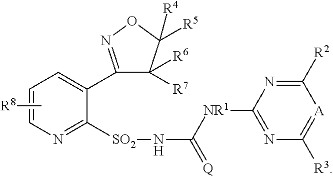

| Ex. | Physical | | | | | | | | | | |
|---|---|---|---|---|---|---|---|---|---|---|---|
| No. | data | R¹ | R² | R³ | R⁴ | R⁶ | R⁵ | R⁷ | R⁸ | Q | A |
| Ia-1 | ¹H-NMR | H | Cl | OCH₃ | H | H | H | H | H | O | CH |
| Ia-2 | ¹H-NMR | H | OCH₃ | OCH₃ | H | H | H | H | H | O | CH |
| Ia-3 | ¹H-NMR | H | CH₃ | CH₃ | H | H | H | H | H | O | CH |
| Ia-4 | ¹H-NMR | H | SCH₃ | OCH₃ | H | H | H | H | H | O | CH |
| Ia-5 | ¹H-NMR | H | H | OCH₃ | H | H | H | H | H | O | CH |
| Ia-6 | ¹H-NMR | H | OCH₃ | OCH₃ | H | H | H | H | H | O | N |
| Ia-7 | ¹H-NMR | H | CH₃ | OCH₃ | H | H | H | H | H | O | N |
| Ia-8 | ¹H-NMR | H | N(CH₃)₂ | OCH₂CF₃ | H | H | H | H | H | O | N |
| Ia-9 | ¹H-NMR | H | CH₃ | OCH₃ | H | H | H | H | H | O | CH |
| Ia-10 | ¹H-NMR | H | OCH₃ | OCH₃ | CH(CH₃)₂ | H | H | H | H | O | CH |
| Ia-11 | ¹H-NMR | H | OCH₃ | OCH₃ | CH₃ | H | H | H | H | O | CH |
| Ia-12 | ¹H-NMR | H | CH₃ | OCH₃ | CH₃ | H | CH₃ | H | H | O | N |
| Ia-13 | ¹H-NMR | H | OCH₃ | OCH₃ | CH₃ | H | CH₃ | H | H | O | CH |
| Ia-14 | ¹H-NMR | H | Cl | OCH₃ | CH₃ | H | CH₃ | H | H | O | CH |
| Ia-15 | ¹H-NMR | H | CH₃ | OCH₃ | CH₃ | H | H | H | H | O | CH |
| Ia-16 | ¹H-NMR | H | Cl | OCH₃ | CH₃ | H | H | H | H | O | CH |
| Ia-17 | ¹H-NMR | H | CH₃ | OCH₃ | CH₂CH₂CH₃ | H | H | H | H | O | CH |
| Ia-18 | ¹H-NMR | H | CH₃ | OCH₃ | CH₂CH₃ | H | H | H | H | O | N |
| Ia-19 | ¹H-NMR | H | CH₃ | OCH₃ | CH₂CH₃ | H | H | H | H | O | CH |
| Ia-20 | ¹H-NMR | H | Cl | OCH₃ | CH₂CH₃ | H | H | H | H | O | CH |
| Ia-21 | ¹H-NMR | H | OCH₃ | OCH₃ | CH₂CH₂CH₃ | H | H | H | H | O | CH |
| Ia-22 | ¹H-NMR | H | CH₃ | OCH₃ | CH(CH₃)₂ | H | H | H | H | O | CH |
| Ia-23 | ¹H-NMR | H | CH₃ | OCH₃ | CH₂CH₂CH₃ | H | H | H | H | O | N |
| Ia-24 | ¹H-NMR | H | OCH₃ | OCH₃ | CH₂CH₃ | H | H | H | H | O | CH |
| Ia-25 | | H | OCH₃ | OCH₃ | H | H | H | H | H | S | CH |
| Ia-26 | | H | OCH₃ | OCH₃ | H | H | H | H | 6-Cl | O | CH |
| Ia-27 | | H | OCH₃ | OCH₃ | H | H | H | H | 6-CH₃ | O | CH |
| Ia-28 | | H | OCH₃ | OCH₃ | H | H | H | H | 6-Br | O | CH |
| Ia-29 | | H | OCH₃ | OCH₃ | H | H | H | H | 6-F | O | CH |
| Ia-30 | | H | OCH₃ | OCH₃ | H | H | H | H | 6-CF₃ | O | CH |
| Ia-31 | | H | OCH₃ | OCH₃ | H | H | H | H | 6-OCH₃ | O | CH |
| Ia-32 | | H | OCH₃ | OCH₃ | H | H | H | H | 6-SCH₃ | O | CH |
| Ia-33 | | H | OCH₃ | OCH₃ | H | H | H | H | 6-SO₂CH₃ | O | CH |
| Ia-34 | | H | OCH₃ | OCH₃ | H | H | H | H | 6-I | O | CH |

| | | | | | | | | | |
|---|---|---|---|---|---|---|---|---|---|
| Ia-35 | H | OCH₃ | OCH₃ | CH₂CH₂CH₂ | H | H | H | O | CH |
| Ia-36 | H | OCH₃ | OCH₃ | CH₂CH₂CH₂CH₂ | H | H | H | O | CH |
| Ia-37 | H | OCH₃ | OCH₃ | CH₂—O—CH₂ | H | H | H | O | CH |
| Ia-38 | H | OCH₃ | OCH₃ | CH₂—S—CH₂ | H | H | H | O | CH |
| Ia-39 | H | OCH₃ | OCH₃ | CH₂—O—CH₂CH₂ | H | H | H | O | CH |
| Ia-40 | H | OCH₃ | OCH₃ | CH₂—S—CH₂CH₂ | H | H | H | O | CH |

¹H NMR data (400 MHz, solvent: CD₃CN, internal standard: tetramethylsilane δ = 0.00 ppm; s = singlet, br. s = broad singlet, d = doublet, dd = double doublet, m = multiplet, q = quartet, t = triplet)

### Ia-1: δ=3.44 (t, 2H); 4.01 (s, 6H); 4.47 (t, 2H); 6.62 (s, 1H); 7.68 (dd, 1H); 7.99 (dd, 1H); 8.39 (br. s, 1H); 8.68 (dd, 1H); 12.2 (br. s, 1H) ppm

### Ia-2: δ=3.44 (t, 2H); 3.94 (s, 6H); 4.46 (t, 2H); 5.87 (s, 1H); 7.67 (dd, 1H); 7.98 (dd, 1H); 8.14 (br. s, 1H); 8.66 (dd, 1H); 12.8 (br. s, 1H) ppm

### Ia-3: δ=2.42 (s, 6H); 3.45 (t, 2H); 4.46 (t, 2H); 6.90 (s, 1H); 7.66 (dd, 1H); 7.97 (dd, 1H); 8.06 (br. s, 1H); 8.66 (dd, 1H); 13.3 (br. s, 1H) ppm

### Ia-4: δ=2.55 (s, 3H); 3.45 (t, 2H); 3.94 (s, 3H); 4.47 (t, 2H); 6.42 (s, 1H); 7.67 (dd, 1H); 7.98 (dd, 1H); 8.18 (br. s, 1H); 8.67 (dd, 1H); 12.9 (br. s, 1H) ppm

### Ia-5: δ=3.45 (t, 2H); 3.97 (s, 3H); 4.46 (t, 2H); 6.54 (d, 1H); 7.66 (dd, 1H); 7.97 (dd, 1H); 8.1-8.4 (br. 1H); 8.29 (d, 1H); 8.66 (dd, 1H); 13.1 (br. s, 1H) ppm

### Ia-6: δ=3.44 (t, 2H); 4.03 (s, 6H); 4.47 (t, 2H); 7.68 (dd, 1H); 7.99 (dd, 1H); 8.4 (br. s, 1H); 8.67 (dd, 1H); 12.5 (br. s, 1H) ppm

### Ia-7: δ=2.49 (s, 3H); 3.44 (t, 2H); 4.02 (s, 3H); 4.47 (t, 2H); 7.68 (dd, 1H); 8.00 (dd, 1H); 8.4 (br., 1H); 8.67 (dd, 1H); 12.6 (br. s, 1H) ppm

### Ia-8: δ=3.16 (s, 3H); 3.19 (s, 3H); 3.44 (t, 2H); 4.47 (t, 2H); 4.87 (q, 2H); 7.67 (dd, 1H); 7.98 (dd, 1H); 8.14 (br. s, 1H); 8.67 (dd, 1H); 12.9 (br. s, 1H) ppm

### Ia-9: δ=3.43 (s, 3H); 3.45 (t, 2H); 3.95 (s, 3H); 4.46 (t, 2H); 6.40 (s, 1H); 7.66 (dd, 1H); 7.97 (dd, 1H); 8.0-8.25 (br., 1H); 8.66 (dd, 1H); 13.4 (br. s, 1H) ppm

### Ia-10: δ=0.95 (d, 3H); 1.01 (d, 3H); 3.18 (dd, 1H); 3.45 (dd, 1H); 3.87 (m, 1H); 3.94 (s, 6H); 4.49 (m, 1H); 5.86 (s, 1H); 7.66 (dd, 1H); 7.97 (dd, 1H); 8.2 (br. s, 1H); 8.65 (dd, 1H); 12.8 (br. s, 1H) ppm

### Ia-11: δ=1.40 (d, 3H); 3.07 (dd, 1H); 3.54 (dd, 1H); 3.94 (s, 6H); 4.89 (m, 1H); 5.87 (s, 1H); 7.66 (dd, 1H); 7.98 (dd, 1H); 8.18 (br. s, 1H); 8.65 (dd, 1H); 12.8 (br. s, 1H) ppm

### Ia-12: δ=1.47 (s, 6H); 2.49 (s, 3H); 3.24 (s, 2H); 4.02 (s, 3H); 7.67 (dd, 1H); 7.99 (dd, 1H); 8.45 (br. s, 1H); 8.64 (dd, 1H); 12.6 (br. s, 1H) ppm

### Ia-13: δ=1.47 (s, 6H); 3.25 (s, 2H); 3.94 (s, 6H); 5.86 (s, 1H); 7.66 (dd, 1H); 7.99 (dd, 1H); 8.16 (br. s, 1H); 8.64 (dd, 1H); 12.8 (br. s, 1H) ppm

### Ia-14: δ=1.47 (s, 6H); 3.25 (s, 2H); 4.00 (s, 3H); 6.61 (s, 1H); 7.67 (dd, 1H); 7.99 (dd, 1H); 8.38 (br. s, 1H); 8.65 (dd, 1H); 12.2 (br. s, 1H) ppm

### Ia-15: δ=1.40 (d, 3H); 2.39 (s, 3H); 3.08 (dd, 1H); 3.55 (dd, 1H); 3.94 (s, 3H); 4.89 (m, 1H); 6.40 (s, 1H); 7.65 (dd, 1H); 7.96 (dd, 1H); 8.0-8.3 (br., 1H); 8.64 (dd, 1H); 13.4 (br., 1H) ppm

### Ia-16: δ=1.40 (d, 3H); 3.07 (dd, 1H); 3.54 (dd, 1H); 4.01 (s, 3H); 4.90 (m, 1H); 6.62 (s, 1H); 7.67 (dd, 1H); 7.99 (dd, 1H); 8.40 (br. s, 1H); 8.66 (dd, 1H); 12.2 (br. s, 1H) ppm

### Ia-17: δ=0.97 (t, 3H); 1.4-1.55 (m, 2H); 1.6-1.7 (m, 1H); 1.72-1.82 (m, 1H); 2.39 (s, 3H); 3.11 (dd, 1H); 3.51 (dd, 1H); 3.95 (s, 3H); 4.75 (m, 1H); 6.40 (s, 1H); 7.65 (dd, 1H); 7.97 (dd, 1H); 8.05-8.25 (br., 1H); 8.64 (dd, 1H); 13.4 (br. s, 1H) ppm

### Ia-18: δ=0.99 (t, 3H); 1.65-1.85 (m, 2H); 2.50 (s, 3H); 3.11 (dd, 1H); 3.49 (dd, 1H); 4.02 (s, 3H); 4.71 (m, 1H); 7.68 (dd, 1H); 7.99 (dd, 1H); 8.42 (br. s, 1H); 8.66 (dd, 1H); 12.6 (br. s, 1H) ppm

### Ia-19: δ=0.98 (t, 3H); 1.65-1.85 (m, 2H); 2.39 (s, 3H); 3.12 (dd, 1H); 3.51 (dd, 1H); 3.95 (s, 3H); 4.70 (m, 1H); 6.40 (s, 1H); 7.65 (dd, 1H); 7.96 (dd, 1H); 8.13 (br. s, 1H); 8.65 (dd, 1H); 13.4 (br. s, 1H) ppm

### Ia-20: δ=0.99 (t, 3H); 1.65-1.85 (m, 2H); 3.11 (dd, 1H); 3.50 (dd, 1H); 4.01 (s, 3H); 4.70 (m, 1H); 6.62 (s, 1H); 7.67 (dd, 1H); 7.98 (dd, 1H); 8.39 (br. s, 1H); 8.66 (dd, 1H); 12.2 (br. s, 1H) ppm

### Ia-21: δ=0.98 (t, 3H); 1.40-1.55 (m, 2H); 1.6-1.7 (m, 1H); 1.7-1.85 (m. 1H); 3.11 (dd, 1H); 3.51 (dd, 1H); 3.94 (s, 6H); 4.75 (m, 1H); 5.86 (s, 1H); 7.66 (dd, 1H); 7.97 (dd, 1H); 8.13 (br. s, 1H); 8.64 (dd, 1H); 12.8 (br. s, 1H) ppm

### Ia-22: δ=0.95 (d, 3H); 1.00 (d, 3H); 2.39 (s, 3H); 3.18 (dd, 1H); 3.46 (dd, 1H); 3.95 (s, 3H); 4.50 (m, 1H); 6.40 (s, 1H); 7.65 (dd, 1H); 7.96 (dd, 1H); 8.05-8.25 (br., 1H); 8.65 (dd, 1H); 13.4 (br. s, 1H) ppm

### Ia-23: δ=0.97 (t, 3H); 1.35-1.55 (m, 2H); 1.6-1.7 (m, 1H); 1.72-1.83 (m, 1H); 2.50 (s, 3H); 3.10 (dd, 1H); 3.50 (dd, 1H); 4.02 (s. 3H); 4.78 (m, 1H); 7.67 (dd, 1H); 7.99 (dd, 1H); 8.44 (br. s, 1H); 8.65 (dd, 1H); 12.6 (br. s, 1H) ppm

### Ia-24: δ=0.98 (t, 3H); 1.63-1.86 (m, 2H); 3.12 (dd, 1H); 3.50 (dd, 1H); 3.94 (s, 6H); 4.70 (m, 1H); 5.87 (s, 1H); 7.66 (dd, 1H); 7.97 (dd, 1H); 8.15 (br. s, 1H); 8.65 (dd, 1H); 12.8 (br. s, 1H) ppm

TABLE 1b
Compounds of the formula (Ib)
(Ib)

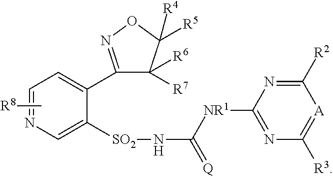

| Ex. No. | R¹ | R² | R³ | R⁴ | R⁶ | R⁵ | R⁷ | R⁸ | Q | A |
|---|---|---|---|---|---|---|---|---|---|---|
| Ib-1 | H | Cl | OCH₃ | H | H | H | H | H | O | CH |
| Ib-2 | H | OCH₃ | OCH₃ | H | H | H | H | H | O | CH |
| Ib-3 | H | CH₃ | CH₃ | H | H | H | H | H | O | CH |
| Ib-4 | H | SCH₃ | OCH₃ | H | H | H | H | H | O | CH |
| Ib-5 | H | H | OCH₃ | H | H | H | H | H | O | CH |
| Ib-6 | H | OCH₃ | OCH₃ | H | H | H | H | H | O | N |
| Ib-7 | H | CH₃ | OCH₃ | H | H | H | H | H | O | N |
| Ib-8 | H | N(CH₃)₂ | OCH₂CF₃ | H | H | H | H | H | O | N |
| Ib-9 | H | CH₃ | OCH₃ | H | H | H | H | H | O | CH |
| Ib-10 | H | OCH₃ | OCH₃ | CH(CH₃)₂ | H | H | H | H | O | CH |
| Ib-11 | H | OCH₃ | OCH₃ | CH₃ | H | H | H | H | O | CH |
| Ib-12 | H | CH₃ | OCH₃ | CH₃ | H | CH₃ | H | H | O | N |
| Ib-13 | H | OCH₃ | OCH₃ | CH₃ | H | CH₃ | H | H | O | CH |
| Ib-14 | H | Cl | OCH₃ | CH₃ | H | CH₃ | H | H | O | CH |
| Ib-15 | H | CH₃ | OCH₃ | CH₃ | H | H | H | H | O | CH |
| Ib-16 | H | Cl | OCH₃ | CH₃ | H | H | H | H | O | CH |
| Ib-17 | H | CH₃ | OCH₃ | CH₂CH₂CH₃ | H | H | H | H | O | CH |
| Ib-18 | H | CH₃ | OCH₃ | CH₂CH₃ | H | H | H | H | O | N |
| Ib-19 | H | CH₃ | OCH₃ | CH₂CH₃ | H | H | H | H | O | CH |
| Ib-20 | H | Cl | OCH₃ | CH₂CH₃ | H | H | H | H | O | CH |
| Ib-21 | H | OCH₃ | OCH₃ | CH₂CH₂CH₃ | H | H | H | H | O | CH |
| Ib-22 | H | CH₃ | OCH₃ | CH(CH₃)₂ | H | H | H | H | O | CH |
| Ib-23 | H | CH₃ | OCH₃ | CH₂CH₂CH₃ | H | H | H | H | O | N |
| Ib-24 | H | OCH₃ | OCH₃ | CH₂CH₃ | H | H | H | H | O | CH |
| Ib-25 | H | OCH₃ | OCH₃ | H | H | H | H | H | S | CH |

| | | | | | | | | | |
|---|---|---|---|---|---|---|---|---|---|
| Ib-26 | H | OCH₃ | OCH₃ | CH₂CH₂CH₂ | H | H | H | O | CH |
| Ib-27 | H | OCH₃ | OCH₃ | CH₂CH₂CH₂CH₂ | H | H | H | O | CH |
| Ib-28 | H | OCH₃ | OCH₃ | CH₂—O—CH₂ | H | H | H | O | CH |
| Ib-29 | H | OCH₃ | OCH₃ | CH₂—S—CH₂ | H | H | H | O | CH |
| Ib-30 | H | OCH₃ | OCH₃ | CH₂—O—CH₂CH₂ | H | H | H | O | CH |
| Ib-31 | H | OCH₃ | OCH₃ | CH₂—S—CH₂CH₂ | H | H | H | O | CH |

TABLE 1c
Compounds of the formuIa (Ic)
(Ic)

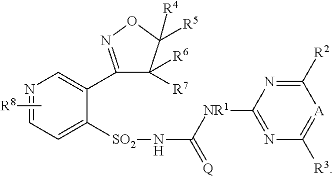

| Ex. No. | R¹ | R² | R³ | R⁴ | R⁶ | R⁵ | R⁷ | R⁸ | Q | A |
|---|---|---|---|---|---|---|---|---|---|---|
| Ic-1 | H | OCH₃ | OCH₃ | H | H | H | H | H | O | CH |
| Ic-2 | H | Cl | OCH₃ | H | H | H | H | H | O | CH |
| Ic-3 | H | CH₃ | CH₃ | H | H | H | H | H | O | CH |
| Ic-4 | H | SCH₃ | OCH₃ | H | H | H | H | H | O | CH |
| Ic-5 | H | H | OCH₃ | H | H | H | H | H | O | CH |
| Ic-6 | H | OCH₃ | OCH₃ | H | H | H | H | H | O | N |
| Ic-7 | H | CH₃ | OCH₃ | H | H | H | H | H | O | N |
| Ic-8 | H | N(CH₃)₂ | OCH₂CF₃ | H | H | H | H | H | O | N |
| Ic-9 | H | CH₃ | OCH₃ | H | H | H | H | H | O | CH |
| Ic-10 | H | OCH₃ | OCH₃ | CH(CH₃)₂ | H | H | H | H | O | CH |
| Ic-11 | H | OCH₃ | OCH₃ | CH₃ | H | H | H | H | O | CH |
| Ic-12 | H | CH₃ | OCH₃ | CH₃ | H | CH₃ | H | H | O | N |
| Ic-13 | H | OCH₃ | OCH₃ | CH₃ | H | CH₃ | H | H | O | CH |
| Ic-14 | H | Cl | OCH₃ | CH₃ | H | CH₃ | H | H | O | CH |
| Ic-15 | H | CH₃ | OCH₃ | CH₃ | H | H | H | H | O | CH |
| Ic-16 | H | Cl | OCH₃ | CH₃ | H | H | H | H | O | CH |
| Ic-17 | H | CH₃ | OCH₃ | CH₂CH₂CH₃ | H | H | H | H | O | CH |
| Ic-18 | H | CH₃ | OCH₃ | CH₂CH₃ | H | H | H | H | O | N |
| Ic-19 | H | CH₃ | OCH₃ | CH₂CH₃ | H | H | H | H | O | CH |
| Ic-20 | H | Cl | OCH₃ | CH₂CH₃ | H | H | H | H | O | CH |
| Ic-21 | H | OCH₃ | OCH₃ | CH₂CH₂CH₃ | H | H | H | H | O | CH |
| Ic-22 | H | CH₃ | OCH₃ | CH(CH₃)₂ | H | H | H | H | O | CH |
| Ic-23 | H | CH₃ | OCH₃ | CH₂CH₂CH₃ | H | H | H | H | O | N |
| Ic-24 | H | OCH₃ | OCH₃ | CH₂CH₃ | H | H | H | H | O | CH |
| Ic-25 | H | OCH₃ | OCH₃ | H | H | H | H | H | S | CH |

| | | | | | | | | | |
|---|---|---|---|---|---|---|---|---|---|
| Ic-26 | H | OCH₃ | OCH₃ | CH₂CH₂CH₂ | H | H | H | O | CH |
| Ic-27 | H | OCH₃ | OCH₃ | CH₂CH₂CH₂CH₂ | H | H | H | O | CH |
| Ic-28 | H | OCH₃ | OCH₃ | CH₂—O—CH₂ | H | H | H | O | CH |
| Ic-29 | H | OCH₃ | OCH₃ | CH₂—S—CH₂ | H | H | H | O | CH |
| Ic-30 | H | OCH₃ | OCH₃ | CH₂—O—CH₂CH₂ | H | H | H | O | CH |
| Ic-31 | H | OCH₃ | OCH₃ | CH₂—S—CH₂CH₂ | H | H | H | O | CH |

TABLE 1d
Compounds of the formula (Id)
(Id)

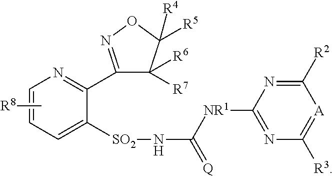

| Ex. No. | R¹ | R² | R³ | R⁴ | R⁶ | R⁵ | R⁷ | R⁸ | Q | A |
|---|---|---|---|---|---|---|---|---|---|---|
| Id-1 | H | OCH₃ | OCH₃ | H | H | H | H | H | O | CH |
| Id-2 | H | Cl | OCH₃ | H | H | H | H | H | O | CH |
| Id-3 | H | CH₃ | CH₃ | H | H | H | H | H | O | CH |
| Id-4 | H | SCH₃ | OCH₃ | H | H | H | H | H | O | CH |
| Id-5 | H | H | OCH₃ | H | H | H | H | H | O | CH |
| Id-6 | H | OCH₃ | OCH₃ | H | H | H | H | H | O | N |
| Id-7 | H | CH₃ | OCH₃ | H | H | H | H | H | O | N |
| Id-8 | H | N(CH₃)₂ | OCH₂CF₃ | H | H | H | H | H | O | N |
| Id-9 | H | CH₃ | OCH₃ | H | H | H | H | H | O | CH |
| Id-10 | H | OCH₃ | OCH₃ | CH(CH₃)₂ | H | H | H | H | O | CH |
| Id-11 | H | OCH₃ | OCH₃ | CH₃ | H | H | H | H | O | CH |
| Id-12 | H | CH₃ | OCH₃ | CH₃ | H | CH₃ | H | H | O | N |
| Id-13 | H | OCH₃ | OCH₃ | CH₃ | H | CH₃ | H | H | O | CH |
| Id-14 | H | Cl | OCH₃ | CH₃ | H | CH₃ | H | H | O | CH |
| Id-15 | H | CH₃ | OCH₃ | CH₃ | H | H | H | H | O | CH |
| Id-16 | H | Cl | OCH₃ | CH₃ | H | H | H | H | O | CH |
| Id-17 | H | CH₃ | OCH₃ | CH₂CH₂CH₃ | H | H | H | H | O | CH |
| Id-18 | H | CH₃ | OCH₃ | CH₂CH₃ | H | H | H | H | O | N |
| Id-19 | H | CH₃ | OCH₃ | CH₂CH₃ | H | H | H | H | O | CH |
| Id-20 | H | Cl | OCH₃ | CH₂CH₃ | H | H | H | H | O | CH |
| Id-21 | H | OCH₃ | OCH₃ | CH₂CH₂CH₃ | H | H | H | H | O | CH |
| Id-22 | H | CH₃ | OCH₃ | CH(CH₃)₂ | H | H | H | H | O | CH |
| Id-23 | H | CH₃ | OCH₃ | CH₂CH₂CH₃ | H | H | H | H | O | N |
| Id-24 | H | OCH₃ | OCH₃ | CH₂CH₃ | H | H | H | H | O | CH |
| Id-25 | H | OCH₃ | OCH₃ | H | H | H | H | H | S | CH |

| | | | | | | | | | |
|---|---|---|---|---|---|---|---|---|---|
| Id-26 | H | OCH₃ | OCH₃ | CH₂CH₂CH₂ | H | H | H | O | CH |
| Id-27 | H | OCH₃ | OCH₃ | CH₂CH₂CH₂CH₂ | H | H | H | O | CH |
| Id-28 | H | OCH₃ | OCH₃ | CH₂—O—CH₂ | H | H | H | O | CH |
| Id-29 | H | OCH₃ | OCH₃ | CH₂—S—CH₂ | H | H | H | O | CH |
| Id-30 | H | OCH₃ | OCH₃ | CH₂—O—CH₂CH₂ | H | H | H | O | CH |
| Id-31 | H | OCH₃ | OCH₃ | CH₂—S—CH₂CH₂ | H | H | H | O | CH |

### 2. 3-(4,5-Dihydroisoxazol-3-yl)pyridine-2-sulfonamide (IIa-1)

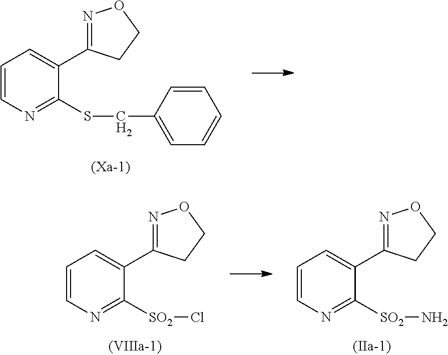

2.7 g (10 mmol) of 2-(benzylthio)-3-(4,5-dihydroisoxazol-3-yl)pyridine (Xa-1) are taken up in a mixture of 50 ml of glacial acetic acid and 25 ml of water. Chlorine gas is passed into the resulting suspension at 0° C. until the mixture turns pale yellow. The clear solution is extracted three times with methylene chloride. The combined organic phases are washed with water and sodium chloride solution, dried with magnesium sulfate and concentrated in vacuo. This gives the compound of the formula (VIIIa-1) as an oily substance, which is employed without further purification in the subsequent reaction.

The crude sulfochloride (VIIIa-1) is taken up in 10 ml of acetonitrile and treated dropwise with 26% strength aqueous ammonia solution until a sample shows a basic reaction. The mixture is concentrated and taken up in methylene chloride. Insoluble salts are filtered off. The filtrate is washed with water, dried and concentrated, and the residue is triturated with diethyl ether. This gives 1.3 g (5.5 mmol) of 3-(4,5-dihydroisoxazol-3-yl)pyridine-2-sulfonamide (IIa-1) of purity (HPLC) 96.1% in the form of colorless crystals, m.p. 155-156° C.

The following can be prepared analogously:

TABLE 2a
Compounds of the formula (VIIIa)
(VIII-a)

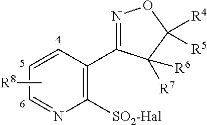

| Ex. | | | | | | |
|---|---|---|---|---|---|---|
| No. | R⁴ | R⁶ | R⁵ | R⁷ | R⁸ | Hal |
| VIIIa-1 | H | H | H | H | H | Cl |
| VIIIa-2 | CH(CH₃)₂ | H | H | H | H | Cl |
| VIIIa-3 | CH₃ | H | H | H | H | Cl |
| VIIIa-4 | CH₃ | H | CH₃ | H | H | Cl |
| VIIIa-5 | CH₂CH₂CH₃ | H | H | H | H | Cl |
| VIIIa-6 | CH₂CH₃ | H | H | H | H | Cl |
| VIIIa-7 | CH₃ | CH₃ | H | H | H | Cl |
| VIIIa-8 | H | H | H | H | 6-Cl | Cl |
| VIIIa-9 | H | H | H | H | 6-CH₃ | Cl |
| VIIIa-10 | H | H | H | H | 6-Br | Cl |
| VIIIa-11 | H | H | H | H | 6-F | Cl |
| VIIIa-12 | H | H | H | H | 6-CF₃ | Cl |
| VIIIa-13 | H | H | H | H | 6-OCH₃ | Cl |
| VIIIa-14 | H | H | H | H | 6-SCH₃ | Cl |
| VIIIa-15 | H | H | H | H | 6-SO₂CH₃ | Cl |
| VIIIa-16 | H | H | H | H | 6-I | Cl |

| | | | | | |
|---|---|---|---|---|---|
| VIIIa-17 | CH₂CH₂CH₂ | H | H | H | Cl |
| VIIIa-18 | CH₂CH₂CH₂CH₂ | H | H | H | Cl |
| VIIIa-19 | CH₂—O—CH₂ | H | H | H | Cl |
| VIIIa-20 | CH₂—S—CH₂ | H | H | H | Cl |
| VIIIa-21 | CH₂—O—CH₂CH₂ | H | H | H | Cl |
| VIIIa-22 | CH₂—S—CH₂CH₂ | H | H | H | Cl |

TABLE 2b
Compounds of the formula (VIIIb)
(VIII-b)

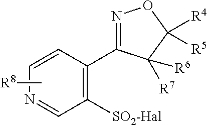

| Ex. | | | | | | |
|---|---|---|---|---|---|---|
| No. | R⁴ | R⁶ | R⁵ | R⁷ | R⁸ | Hal |
| VIIIb-1 | H | H | H | H | H | Cl |
| VIIIb-2 | CH(CH₃)₂ | H | H | H | H | Cl |
| VIIIb-3 | CH₃ | H | H | H | H | Cl |
| VIIIb-4 | CH₃ | H | CH₃ | H | H | Cl |
| VIIIb-5 | CH₂CH₂CH₃ | H | H | H | H | Cl |
| VIIIb-6 | CH₂CH₃ | H | H | H | H | Cl |
| VIIIb-7 | CH₃ | CH₃ | H | H | H | Cl |

| | | | | | |
|---|---|---|---|---|---|
| VIIIb-8 | CH₂CH₂CH₂ | H | H | H | Cl |
| VIIIb-9 | CH₂CH₂CH₂CH₂ | H | H | H | Cl |
| VIIIb-10 | CH₂—O—CH₂ | H | H | H | Cl |
| VIIIb-11 | CH₂—S—CH₂ | H | H | H | Cl |
| VIIIb-12 | CH₂—O—CH₂CH₂ | H | H | H | Cl |
| VIIIb-13 | CH₂—S—CH₂CH₂ | H | H | H | Cl |

TABLE 2c
Compounds of the formula (VIIIc)
(VIII-c)

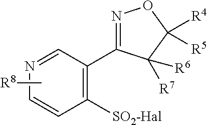

| Ex. | | | | | | |
|---|---|---|---|---|---|---|
| No. | R⁴ | R⁶ | R⁵ | R⁷ | R⁸ | Hal |
| VIIIc-1 | H | H | H | H | H | Cl |
| VIIIc-2 | CH(CH₃)₂ | H | H | H | H | Cl |
| VIIIc-3 | CH₃ | H | H | H | H | Cl |
| VIIIc-4 | CH₃ | H | CH₃ | H | H | Cl |
| VIIIc-5 | CH₂CH₂CH₃ | H | H | H | H | Cl |
| VIIIc-6 | CH₂CH₃ | H | H | H | H | Cl |
| VIIIc-7 | CH₃ | CH₃ | H | H | H | Cl |

| | | | | | |
|---|---|---|---|---|---|
| VIIIc-8 | CH₂CH₂CH₂ | H | H | H | Cl |
| VIIIc-90 | CH₂CH₂CH₂CH₂ | H | H | H | Cl |
| VIIIc-10 | CH₂—O—CH₂ | H | H | H | Cl |
| VIIIc-11 | CH₂—S—CH₂ | H | H | H | Cl |
| VIIIc-12 | CH₂—O—CH₂CH₂ | H | H | H | Cl |
| VIIIc-13 | CH₂—S—CH₂CH₂ | H | H | H | Cl |

TABLE 2d
Compounds of the formula (VIIId)
(VIII-d)

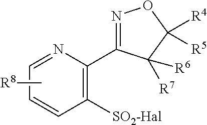

| Ex. | | | | | | |
|---|---|---|---|---|---|---|
| No. | R⁴ | R⁶ | R⁵ | R⁷ | R⁸ | Hal |
| VIIId-1 | H | H | H | H | H | Cl |
| VIIId-2 | CH(CH₃)₂ | H | H | H | H | Cl |
| VIIId-3 | CH₃ | H | H | H | H | Cl |
| VIIId-4 | CH₃ | H | CH₃ | H | H | Cl |
| VIIId-5 | CH₂CH₂CH₃ | H | H | H | H | Cl |
| VIIId-6 | CH₂CH₃ | H | H | H | H | Cl |
| VIIId-7 | CH₃ | CH₃ | H | H | H | Cl |

| | | | | | |
|---|---|---|---|---|---|
| VIIId-8 | CH₂CH₂CH₂ | H | H | H | Cl |
| VIIId-9 | CH₂CH₂CH₂CH₂ | H | H | H | Cl |
| VIIId-10 | CH₂—O—CH₂ | H | H | H | Cl |
| VIIId-11 | CH₂—S—CH₂ | H | H | H | Cl |
| VIIId-12 | CH₂—O—CH₂CH₂ | H | H | H | Cl |
| VIIId-13 | CH₂—S—CH₂CH₂ | H | H | H | Cl |

TABLE 3a
Compounds of the formula (IIa)
(IIa)

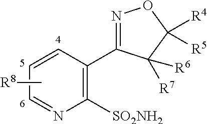

| Ex. | Physical data | | | | | |
|---|---|---|---|---|---|---|
| No. | m.p. [° C.] | R⁴ | R⁶ | R⁵ | R⁷ | R⁸ |
| IIa-1 | 155-156 | H | H | H | H | H |
| IIa-2 | 154-155 | CH(CH₃)₂ | H | H | H | H |
| IIa-3 | 152-153 | CH₃ | H | H | H | H |
| IIa-4 | 201-202 | CH₃ | H | CH₃ | H | H |
| IIa-5 | 139-140 | CH₂CH₂CH₃ | H | H | H | H |
| IIa-6 | 173-174 | CH₂CH₃ | H | H | H | H |
| IIa-7 | | CH₃ | CH₃ | H | H | H |
| IIa-8 | | H | H | H | H | 6-Cl |
| IIa-9 | | H | H | H | H | 6-CH₃ |
| IIa-10 | | H | H | H | H | 6-Br |
| IIa-11 | | H | H | H | H | 6-F |
| IIa-12 | | H | H | H | H | 6-CF₃ |
| IIa-13 | | H | H | H | H | 6-OCH₃ |
| IIa-14 | | H | H | H | H | 6-SCH₃ |
| IIa-15 | | H | H | H | H | 6-SO₂CH₃ |
| IIa-16 | | H | H | H | H | 6-I |

| | | | | |
|---|---|---|---|---|
| IIa-17 | CH₂CH₂CH₂ | H | H | H |
| IIa-18 | CH₂CH₂CH₂CH₂ | H | H | H |
| IIa-19 | CH₂—O—CH₂ | H | H | H |
| IIa-20 | CH₂—S—CH₂ | H | H | H |
| IIa-21 | CH₂—O—CH₂CH₂ | H | H | H |
| IIa-22 | CH₂—S—CH₂CH₂ | H | H | H |

TABLE 3b
Compounds of the formula (IIb)

(IIb)

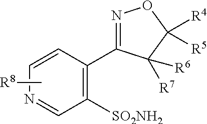

| Ex. No. | R⁴ | R⁶ | R⁵ | R⁷ | R⁸ |
|---|---|---|---|---|---|
| IIb-1 | H | H | H | H | H |
| IIb-2 | CH(CH₃)₂ | H | H | H | H |
| IIb-3 | CH₃ | H | H | H | H |
| IIb-4 | CH₃ | H | CH₃ | H | H |
| IIb-5 | CH₂CH₂CH₃ | H | H | H | H |
| IIb-6 | CH₂CH₃ | H | H | H | H |
| IIb-7 | CH₃ | CH₃ | H | H | H |

| | | | | |
|---|---|---|---|---|
| IIb-8 | CH₂CH₂CH₂ | H | H | H |
| IIb-9 | CH₂CH₂CH₂CH₂ | H | H | H |
| IIb-10 | CH₂—O—CH₂ | H | H | H |
| IIb-11 | CH₂—S—CH₂ | H | H | H |
| IIb-12 | CH₂—O—CH₂CH₂ | H | H | H |
| IIb-13 | CH₂—S—CH₂CH₂ | H | H | H |

TABLE 3c
Compounds of the formula (IIc)

(IIc)

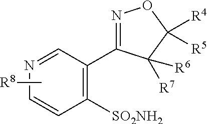

| | Physical | | | | | |
|---|---|---|---|---|---|---|
| Ex. No. | data | R⁴ | R⁶ | R⁵ | R⁷ | R⁸ |
| IIc-1 | | H | H | H | H | H |
| IIc-2 | | CH(CH₃)₂ | H | H | H | H |
| IIc-3 | | CH₃ | H | H | H | H |
| IIc-4 | | CH₃ | H | CH₃ | H | H |
| IIc-5 | | CH₂CH₂CH₃ | H | H | H | H |
| IIc-6 | | CH₂CH₃ | H | H | H | H |
| IIc-7 | | CH₃ | CH₃ | H | H | H |

| | | | | |
|---|---|---|---|---|
| IIc-8 | CH₂CH₂CH₂ | H | H | H |
| IIc-9 | CH₂CH₂CH₂CH₂ | H | H | H |
| IIc-10 | CH₂—O—CH₂ | H | H | H |
| IIc-11 | CH₂—S—CH₂ | H | H | H |
| IIc-12 | CH₂—O—CH₂CH₂ | H | H | H |
| IIc-13 | CH₂—S—CH₂CH₂ | H | H | H |

TABLE 3d
Compounds of the formula (IId)

(IId)

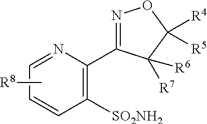

| Ex. No. | R⁴ | R⁶ | R⁵ | R⁷ | R⁸ |
|---|---|---|---|---|---|
| IId-1 | H | H | H | H | H |
| IId-2 | CH(CH₃)₂ | H | H | H | H |
| IId-3 | CH₃ | H | H | H | H |
| IId-4 | CH₃ | H | CH₃ | H | H |
| IId-5 | CH₂CH₂CH₃ | H | H | H | H |
| IId-6 | CH₂CH₃ | H | H | H | H |
| IId-7 | CH₃ | CH₃ | H | H | H |

| | | | | |
|---|---|---|---|---|
| IId-8 | CH₂CH₂CH₂ | H | H | H |
| IId-9 | CH₂CH₂CH₂CH₂ | H | H | H |
| IId-10 | CH₂—O—CH₂ | H | H | H |
| IId-11 | CH₂—S—CH₂ | H | H | H |
| IId-12 | CH₂—O—CH₂CH₂ | H | H | H |
| IId-13 | CH₂—S—CH₂CH₂ | H | H | H |

### 3. 2-(Benzylthio)-3-(4,5-dihydroisoxazol-3-yl)pyridine (Xa-1)

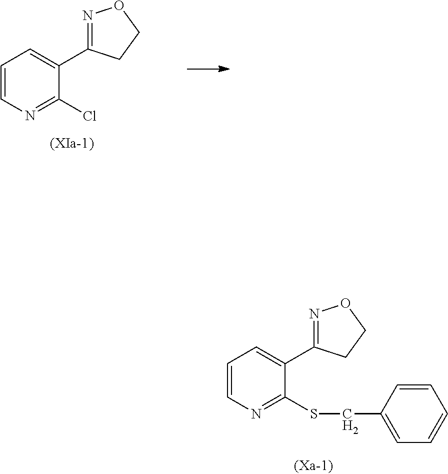

2.86 g (0.11 mol) of sodium hydride (95%) are suspended in 600 ml of absolute N,N-dimethylformamide. 13.64 g (0.11 mol) of benzyl mercaptan are added dropwise with stirring, during which process the temperature of the mixture rises.

18.3 g (0.1 mol) of 2-chloro-3-(4,5-dihydroisoxazol-3-yl)pyridine (XIa-1), dissolved in 30 ml of N,N-dimethylformamide, are added portionwise to the clear solution, and stirring is continued for 3 hours at 20° C. The solution is concentrated, and the residue is taken up in methylene chloride and the mixture is washed repeatedly with water. After drying with magnesium sulfate, filtration and concentration, the residue is crystallized in diisopropyl ether. This gives 23.8 g (87.4 mmol) of 2-(benzylthio)-3-(4,5-dihydroisoxazol-3-yl)pyridine (Xa-1) of purity (HPLC) 99.3% in the form of colorless crystals, m.p. 97-98° C.

The following can be prepared analogously:

TABLE 4a
Compounds of the formula (Xa)

(Xa)

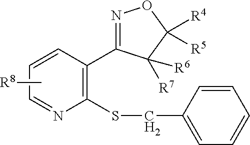

| | Physical data | | | | | |
|---|---|---|---|---|---|---|
| Ex. No. | m.p. [° C.] | R⁴ | R⁶ | R⁵ | R⁷ | R⁸ |
| Xa-1 | 97-98 | H | H | H | H | H |
| Xa-2 | 89-90 | CH₃ | H | H | H | H |
| Xa-3 | 53-54 | CH(CH₃)₂ | H | H | H | H |
| Xa-4 | 115-116 | CH₃ | H | CH₃ | H | H |
| Xa-5 | 106-108 | CH₂CH₂CH₃ | H | H | H | H |
| Xa-6 | ¹H-NMR | CH₂CH₃ | H | H | H | H |
| Xa-7 | | CH₃ | CH₃ | H | H | H |
| Xa-8 | | H | H | H | H | 6-Cl |
| Xa-9 | | H | H | H | H | 6-CH₃ |
| Xa-10 | | H | H | H | H | 6-Br |
| Xa-11 | | H | H | H | H | 6-F |
| Xa-12 | | H | H | H | H | 6-CF₃ |
| Xa-13 | | H | H | H | H | 6-OCH₃ |
| Xa-14 | | H | H | H | H | 6-SCH₃ |
| Xa-15 | | H | H | H | H | 6-SO₂CH₃ |
| Xa-16 | | H | H | H | H | 6-I |

| | | | | |
|---|---|---|---|---|
| Xa-17 | CH₂CH₂CH₂ | H | H | H |
| Xa-18 | CH₂CH₂CH₂CH₂ | H | H | H |
| Xa-19 | CH₂—O—CH₂ | H | H | H |
| Xa-20 | CH₂—S—CH₂ | H | H | H |
| Xa-21 | CH₂—O—CH₂CH₂ | H | H | H |
| Xa-22 | CH₂—S—CH₂CH₂ | H | H | H |

¹H NMR data (400 MHz, solvent: CD₃CN, internal standard: tetramethylsilane
δ = 0.00 ppm; s = singlet, br. s = broad singlet, d = doublet, dd = double doublet, m = multiplet, q = quartet, t = triplet)

### Xa-6: δ=0.96 (t, 3H); 1.6-1.8 (m, 2H); 3.02 (dd, 1H); 3.41 (dd, 1H); 4.45 (s, 2H); 4.62 (m, 1H); 7.14 (dd, 1H); 7.15-7.33 (m, 3H); 7.40-7.48 (m, 2H); 7.65 (dd, 1H); 8.45 (dd, 1H) ppm

TABLE 4b
Compounds of the formula (Xb)

(Xb)

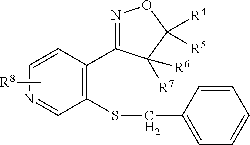

| Ex. No. | R⁴ | R⁶ | R⁵ | R⁷ | R⁸ |
|---|---|---|---|---|---|
| Xb-1 | H | H | H | H | H |
| Xb-2 | CH₃ | H | H | H | H |
| Xb-3 | CH(CH₃)₂ | H | H | H | H |
| Xb-4 | CH₃ | H | CH₃ | H | H |
| Xb-5 | CH₂CH₂CH₃ | H | H | H | H |
| Xb-6 | CH₂CH₃ | H | H | H | H |
| Xb-7 | CH₃ | CH₃ | H | H | H |

| | | | | |
|---|---|---|---|---|
| Xb-8 | CH₂CH₂CH₂ | H | H | H |
| Xb-9 | CH₂CH₂CH₂CH₂ | H | H | H |
| Xb-10 | CH₂—O—CH₂ | H | H | H |
| Xb-11 | CH₂—S—CH₂ | H | H | H |
| Xb-12 | CH₂—O—CH₂CH₂ | H | H | H |
| Xb-13 | CH₂—S—CH₂CH₂ | H | H | H |

TABLE 4c
Compounds of the formula (Xc)

(Xc)

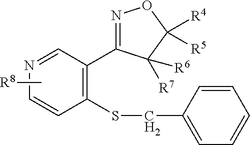

| Ex. No. | R⁴ | R⁶ | R⁵ | R⁷ | R⁸ |
|---|---|---|---|---|---|
| Xc-1 | H | H | H | H | H |
| Xc-2 | CH₃ | H | H | H | H |
| Xc-3 | CH(CH₃)₂ | H | H | H | H |
| Xc-4 | CH₃ | H | CH₃ | H | H |
| Xc-5 | CH₂CH₂CH₃ | H | H | H | H |
| Xc-6 | CH₂CH₃ | H | H | H | H |
| Xc-7 | CH₃ | CH₃ | H | H | H |

| | | | | |
|---|---|---|---|---|
| Xc-8 | CH₂CH₂CH₂ | H | H | H |
| Xc-9 | CH₂CH₂CH₂CH₂ | H | H | H |
| Xc-10 | CH₂—O—CH₂ | H | H | H |
| Xc-11 | CH₂—S—CH₂ | H | H | H |
| Xc-12 | CH₂—O—CH₂CH₂ | H | H | H |
| Xc-13 | CH₂—S—CH₂CH₂ | H | H | H |

TABLE 4d
Compounds of the formula (Xd)

(Xd)

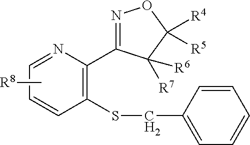

| Ex. No. | R⁴ | R⁶ | R⁵ | R⁷ | R⁸ |
|---|---|---|---|---|---|
| Xd-1 | H | H | H | H | H |
| Xd-2 | CH₃ | H | H | H | H |
| Xd-3 | CH(CH₃)₂ | H | H | H | H |
| Xd-4 | CH₃ | H | CH₃ | H | H |
| Xd-5 | CH₂CH₂CH₃ | H | H | H | H |
| Xd-6 | CH₂CH₃ | H | H | H | H |
| Xd-7 | CH₃ | CH₃ | H | H | H |

| | | | | |
|---|---|---|---|---|
| Xd-8 | CH₂CH₂CH₂ | H | H | H |
| Xd-9 | CH₂CH₂CH₂CH₂ | H | H | H |
| Xd-10 | CH₂—O—CH₂ | H | H | H |
| Xd-11 | CH₂—S—CH₂ | H | H | H |
| Xd-12 | CH₂—O—CH₂CH₂ | H | H | H |
| Xd-13 | CH₂—S—CH₂CH₂ | H | H | H |

### 4. 2-Chloro-3-(4,5-dihydroisoxazol-3-yl)pyridine (XIa-1)

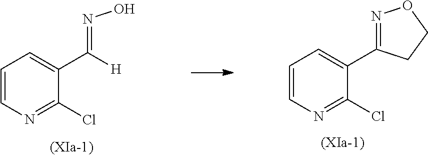

In a pressure vessel, 22.5 g (0.14 mol) of 2-chloronicotinaldehyde oxime are dissolved in 1650 ml of methylene chloride. The autoclave is sealed and made inert with nitrogen. Ethylene is charged until 5 bar is reached. 279 g (231.3 ml) of sodium hypochlorite solution (10-13% strength) are pumped in at 20° C. in the course of 1 hour, with thorough stirring. After everything has been metered in, stirring is continued for 15 hours at room temperature. After the pressure of the pressure vessel has been released, excess ethylene is purged with nitrogen. The organic phase is separated off, washed with water and dried with Baylith TE 144. After the solvent has been removed in vacuo, 23.8 g of 2-chloro-3-(4,5-dihydroisoxazol-3-yl)pyridine (XIa-1) of purity (HPLC) 98.1% are obtained, logp=0.97.

The following can be prepared analogously:

TABLE 5a
Compounds of the formula (XIa)

(XIa)

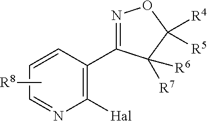

| | Physical data | | | | | | |
|---|---|---|---|---|---|---|---|
| Ex. No. | m.p. [° C.] | R⁴ | R⁶ | R⁵ | R⁷ | R⁸ | Hal |
| XIa-1 | 183-185 | H | H | H | H | H | Cl |
| XIa-2 | ¹H-NMR | CH₃ | H | H | H | H | Cl |
| XIa-3 | ¹H-NMR | CH(CH₃)₂ | H | H | H | H | Cl |
| XIa-4 | 48-50 | CH₃ | H | CH₃ | H | H | Cl |
| XIa-5 | 131-133 | CH₂CH₂CH₃ | H | H | H | H | Cl |
| XIa-6 | 132-133 | CH₂CH₃ | H | H | H | H | Cl |
| XIa-7 | 160-161 | CH₃ | CH₃ | H | H | H | Cl |
| XIa-8 | | H | H | H | H | 6-Cl | Cl |
| XIa-9 | | H | H | H | H | 6-CH₃ | Cl |
| XIa-10 | | H | H | H | H | 6-Br | Cl |
| XIa-11 | | H | H | H | H | 6-F | Cl |
| XIa-12 | | H | H | H | H | 6-CF₃ | Cl |
| XIa-13 | | H | H | H | H | 6-OCH₃ | Cl |
| XIa-14 | | H | H | H | H | 6-SCH₃ | Cl |
| XIa-15 | | H | H | H | H | 6-SO₂CH₃ | Cl |
| XIa-16 | | H | H | H | H | 6-I | Cl |

| | | | | | |
|---|---|---|---|---|---|
| XIa-17 | CH₂CH₂CH₂ | H | H | H | Cl |
| XIa-18 | CH₂CH₂CH₂CH₂ | H | H | H | Cl |
| XIa-19 | CH₂—O—CH₂ | H | H | H | Cl |
| XIa-20 | CH₂—S—CH₂ | H | H | H | Cl |
| XIa-21 | CH₂—O—CH₂CH₂ | H | H | H | Cl |
| XIa-22 | CH₂—S—CH₂CH₂ | H | H | H | Cl |

¹H NMR data (400 MHz, solvent: CD₃CN, internal standard: tetramethylsilane
δ = 0.00 ppm; s = singlet, br. s = broad singlet, d = doublet, dd = double doublet, m = multiplet, q = quartet, t = triplet)

### XIa-2: δ=1.38 (d, 3H); 3.07 (dd, 1H); 3.54 (dd, 1H); 4.88 (m, 1H); 7.38 (dd, 1H); 7.96 (dd, 1H); 8.40 (dd, 1H) ppm

### XIa-3: δ=0.95 (d, 3H); 0.99 (d. 3H); 3.19 (dd, 1H); 3.44 (dd, 1H); 4.52 (m, 1H); 7.38 (dd, 1H); 7.94 (dd, 1H); 8.40 (dd, 1H) ppm

TABLE 5b
Compounds of the formula (XIb)

(XIb)

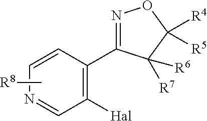

| Ex. No. | R⁴ | R⁶ | R⁵ | R⁷ | R⁸ | Hal |
|---|---|---|---|---|---|---|
| XIb-1 | H | H | H | H | H | Cl |
| XIb-2 | CH₃ | H | H | H | H | Cl |
| XIb-3 | CH(CH₃)₂ | H | H | H | H | Cl |
| XIb-4 | CH₃ | H | CH₃ | H | H | Cl |
| XIb-5 | CH₂CH₂CH₃ | H | H | H | H | Cl |
| XIb-6 | CH₂CH₃ | H | H | H | H | Cl |
| XIb-7 | CH₃ | CH₃ | H | H | H | Cl |

| | | | | | |
|---|---|---|---|---|---|
| XIb-8 | CH₂CH₂CH₂ | H | H | H | Cl |
| XIb-9 | CH₂CH₂CH₂CH₂ | H | H | H | Cl |
| XIb-10 | CH₂—O—CH₂ | H | H | H | Cl |
| XIb-11 | CH₂—S—CH₂ | H | H | H | Cl |
| XIb-12 | CH₂—O—CH₂CH₂ | H | H | H | Cl |
| XIb-13 | CH₂—S—CH₂CH₂ | H | H | H | Cl |

TABLE 5c
The compounds of the formula (XIc)
(XIc)

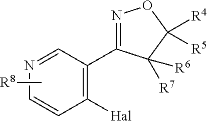

| Ex. No. | R⁴ | R⁶ | R⁵ | R⁷ | R⁸ | Hal |
|---|---|---|---|---|---|---|
| XIc-1 | H | H | H | H | H | Cl |
| XIc-2 | CH₃ | H | H | H | H | Cl |
| XIc-3 | CH(CH₃)₂ | H | H | H | H | Cl |
| XIc-4 | CH₃ | H | CH₃ | H | H | Cl |
| XIc-5 | CH₂CH₂CH₃ | H | H | H | H | Cl |
| XIc-6 | CH₂CH₃ | H | H | H | H | Cl |
| XIc-7 | CH₃ | CH₃ | H | H | H | Cl |

| | | | | | |
|---|---|---|---|---|---|
| XIc-8 | CH₂CH₂CH₂ | H | H | H | Cl |
| XIc-9 | CH₂CH₂CH₂CH₂ | H | H | H | Cl |
| XIc-10 | CH₂—O—CH₂ | H | H | H | Cl |
| XIc-11 | CH₂—S—CH₂ | H | H | H | Cl |
| XIc-12 | CH₂—O—CH₂CH₂ | H | H | H | Cl |
| XIc-13 | CH₂—S—CH₂CH₂ | H | H | H | Cl |

TABLE 5d
Compounds of the formula (XId)

(XId)

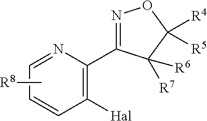

| | Physical | | | | | | |
|---|---|---|---|---|---|---|---|
| Ex. No. | data | R⁴ | R⁶ | R⁵ | R⁷ | R⁸ | Hal |
| XId-1 | ¹H-NMR | H | H | H | H | H | F |
| XId-2 | | CH₃ | H | H | H | H | F |
| XId-3 | | CH(CH₃)₂ | H | H | H | H | F |
| XId-4 | | CH₃ | H | CH₃ | H | H | F |
| XId-5 | | CH₂CH₂CH₃ | H | H | H | H | F |
| XId-6 | | CH₂CH₃ | H | H | H | H | F |
| XId-7 | | CH₃ | CH₃ | H | H | H | F |

| | | | | | |
|---|---|---|---|---|---|
| XId-8 | CH₂CH₂CH₂ | H | H | H | F |
| XId-9 | CH₂CH₂CH₂CH₂ | H | H | H | F |
| XId-10 | CH₂—O—CH₂ | H | H | H | F |
| XId-11 | CH₂—S—CH₂ | H | H | H | F |
| XId-12 | CH₂—O—CH₂CH₂ | H | H | H | F |
| XId-13 | CH₂—S—CH₂CH₂ | H | H | H | F |

1H NMR data (400 MHz, solvent: CD₃CN, internal standard: tetramethylsilane
δ = 0.00 ppm; s = singlet, br. s = broad singlet, d = doublet, dd = double doublet, m = multiplet, q = quartet, t = triplet)

### XId-1: δ=3.47 (t, 2H); 4.40 (t, 2H); 7.39-7.44 (m, 1H); 7.58-7.63 (m, 1H); 8.46 (m, 1H) ppm

### B. FORMULATION EXAMPLES

a) A dust is obtained by mixing 10 parts by weight of a compound of the formula (I) and 90 parts by weight of talc as inert substance and comminuting the mixture in a hammer mill.
b) A wettable powder which is readily dispersible in water is obtained by mixing 25 parts by weight of a compound of the formula (I), 64 parts by weight of kaolin-containing quartz as inert substance, 10 parts by weight of potassium lignosulfonate and 1 part by weight of sodium oleoylmethyltaurinate as wetting agent and dispersant and grinding the mixture in a pinned-disk mill.
c) A dispersion concentrate which is readily dispersible in water is obtained by mixing 20 parts by weight of a compound of the formula (I) with 6 parts by weight of alkylphenol polyglycol ether (®Triton X 207), 3 parts by weight of isotridecanol polyglycol ether (8 EO) and 71 parts by weight of paraffinic mineral oil (boiling range for example approximately 255 to above 277° C.) and grinding the mixture in a ball mill to a fineness of below 5 micrometers.
d) An emulsifiable concentrate is obtained from 15 parts by weight of a compound of the formula (I), 75 parts by weight of cyclohexanone as solvent and 10 parts by weight of oxethylated nonylphenol as emulsifier.
e) Water-dispersible granules are obtained by mixing
   75 parts by weight of a compound of the formula (I),
   10 ″ of calcium lignosulfonate,
   5 ″ of sodium lauryl sulfate,
   3 ″ of polyvinyl alcohol and
   7 ″ of kaolin,
   grinding the mixture in a pinned-disk mill and granulating the powder in a fluidized bed by spraying on water as granulation liquid.
f) Water-dispersible granules are also obtained by homogenizing and precomminuting, in a colloid mill,
   25 parts by weight of a compound of the formula (I),
   5 ″ of sodium 2,2′-dinaphthylmethane-6,6′-disulfonate,
   2 ″ of sodium oleoylmethyltaurinate,
   1 part by weight of polyvinyl alcohol,
   17 parts by weight of calcium carbonate and
   50 ″ of water,
   then grinding the mixture in a bead mill and atomizing and drying the resulting suspension in a spray tower by means of a single-substance nozzle.

### C. BIOLOGICAL EXAMPLES

### 1. Pre-Emergence Herbicidal Activity/Crop Plant Tolerance

Seeds of monocotyledonous or dicotyledonous weed plants or crop plants are placed in sandy loam in wood fiber pots and covered with soil. The compounds according to the invention, which are formulated as wettable powders (WP) are then applied to the surface of the soil cover in the form of an aqueous suspension or emulsion with a water application rate of 600 I/ha (converted), with addition of 0.2% wetter.

After the treatment, the pots are placed in the greenhouse and maintained under good growth conditions for the test plants. After approximately 3 weeks, the activity of the preparation is scored visually in comparison with untreated controls (herbicidal activity in percent (%): 100% activity=plants have died, 0% activity=like control plants).

As demonstrated by the results, compounds according to the invention show good herbicidal pre-emergence activity against a broad spectrum of harmful grasses and harmful broad-leaved weeds. For example, the compounds according to the invention have very good herbicidal activity against harmful plants such as, for example, Alopecurus myosuroides, Matricaria inodora and Stellaria media when applied by the pre-emergence method at an application rate of 0.08 kg and less of active substance per hectare.

The following results were achieved when using the compounds according to the invention pre-emergence:

| Compound | Dosage rate | Unit | ALOMY | MATIN | STEME |
|---|---|---|---|---|---|
| Ia-1 | 80 | g/ha | 90 | 90 | 90 |
| Ia-2 | 80 | g/ha | 90 | 90 | 90 |
| Ia-3 | 80 | g/ha | 80 | 80 | 80 |
| Ia-4 | 80 | g/ha | 80 | 90 | 90 |
| Ia-9 | 80 | g/ha | | | 80 |
| Ia-10 | 80 | g/ha | 80 | 80 | 80 |
| Ia-11 | 80 | g/ha | 90 | 80 | 80 |
| Ia-13 | 80 | g/ha | 90 | 80 | 80 |
| Ia-15 | 80 | g/ha | 80 | 80 | |
| Ia-16 | 80 | g/ha | 80 | 80 | |
| Ia-17 | 80 | g/ha | 90 | | |
| Ia-19 | 80 | g/ha | 80 | | 80 |
| Ia-20 | 80 | g/ha | 80 | | |
| Ia-21 | 80 | g/ha | | 80 | 80 |
| Ia-22 | 80 | g/ha | | | 100 |
| Ia-23 | 80 | g/ha | | 80 | |
| Ia-24 | 80 | g/ha | 90 | 90 | 90 |

In the table, the individual crops are abbreviated as follows:

ALOMY: Slender meadow foxtail (Alopecurus myosuroides)
MATIN: Scentless camomile (Matricaria inodora)
STEME: Chickweed (Stellaria media)

### 2. Post-Emergence Herbicidal Activity

Seeds of monocotyledonous or dicotyledonous weed plants or crop plants are placed in sandy loam in wood fiber pots, covered with soil and grown in the greenhouse under good growth conditions. 2 to 3 weeks after sowing, the test plants are treated in the one-leaf stage. The compounds according to the invention, which are formulated as wettable powders (WP), are then sprayed onto the green plant parts in the form of an aqueous suspension or emulsion with a water application rate of 600 I/ha (converted) with addition of 0.2% of wetter. After the test plants have been left to stand in the greenhouse for approximately 3 weeks under optimal growth conditions, the activity of the preparation is scored visually in comparison with untreated controls (herbicidal activity in percent (%): 100% activity=plants have died, 0% activity=like control plants).

As demonstrated by the results, compounds according to the invention show good herbicidal post-emergence activity against a broad spectrum of harmful grasses and harmful broad-leaved weeds. For example, the compounds according to the invention have very good herbicidal activity against harmful plants such as, for example, Alopecurus myosuroides, Echinochloa crus-galli, Lolium multiflorum, Abutilon theophrasti, Amaranthus retroflexus, Pharbitis purpurea, Stellaria media and Viola tricolor when applied by the post-emergence method at an application rate of 0.08 kg and less of active substance per hectare.

The following results were achieved when using the compounds according to the invention post-emergence:

| | Dosage | | | | | | | | | |
|---|---|---|---|---|---|---|---|---|---|---|
| Compound | rate | Unit | ALOMY | ECHCG | LOLMU | ABUTH | AMARE | PHBPU | STEME | VIOTR |
| Ia-1 | 80 | g/ha | 80 | 100 | 90 | 90 | 90 | 100 | 90 | 80 |
| Ia-2 | 80 | g/ha | 80 | 100 | 90 | 100 | 90 | 90 | 90 | 90 |
| Ia-3 | 80 | g/ha | 80 | 90 | 80 | 90 | 100 | 90 | 90 | 80 |
| Ia-4 | 80 | g/ha | 80 | 90 | | 80 | 100 | 100 | 90 | 80 |
| Ia-6 | 80 | g/ha | | | 90 | | 80 | | | |
| Ia-7 | 80 | g/ha | 80 | | 90 | 80 | | | | |
| Ia-8 | 80 | g/ha | | 80 | 90 | 80 | | | | |
| Ia-9 | 80 | g/ha | 80 | | 90 | 80 | | 90 | 90 | |
| Ia-10 | 80 | g/ha | 90 | 100 | 90 | 90 | 100 | 80 | 90 | 90 |
| Ia-11 | 80 | g/ha | 90 | 100 | 100 | 90 | 100 | 90 | 100 | 90 |
| Ia-13 | 80 | g/ha | 90 | 90 | 90 | 90 | 90 | 90 | 100 | 90 |
| Ia-14 | 80 | g/ha | | 80 | | | 90 | | 80 | 90 |
| Ia-15 | 80 | g/ha | 90 | 80 | 80 | | 90 | | 80 | 80 |
| Ia-16 | 80 | g/ha | 80 | 80 | 90 | | 90 | | 100 | 80 |
| Ia-17 | 80 | g/ha | 80 | | 80 | | 80 | | | |
| Ia-18 | 80 | g/ha | | 80 | | | | | | |
| Ia-19 | 80 | g/ha | 90 | 80 | 80 | | 80 | 80 | 80 | 80 |
| Ia-20 | 80 | g/ha | 80 | 80 | 80 | | 80 | | 80 | 80 |
| Ia-21 | 80 | g/ha | 80 | 80 | 80 | 80 | 80 | 80 | 80 | 80 |
| Ia-22 | 80 | g/ha | 80 | | 80 | 80 | | 80 | | 80 |
| Ia-24 | 80 | g/ha | | 100 | | 80 | 80 | 90 | 90 | |

In the table, the individual crops are abbreviated as follows:

ALOMY: Slender meadow foxtail (Alopecurus myosuroides)
ECHCG: Barnyard grass (Echinochloa crus-galli)
LOLMU: Italian rye grass (Lolium multiflorum)
ABUTH: Velvet leaf (Abutilon theophrasti)
AMARE: Redroot pigweed (Amaranthus retroflexus)
PHBPU: Purple morning glory (Pharbitis/Ipomoea purpurea)
STEME: Chickweed (Stellaria media)
VIOTR: Wild pansy (Viola tricolor)

1. An N-azinyl-N′-pyridylsulfonylurea of formula (I) 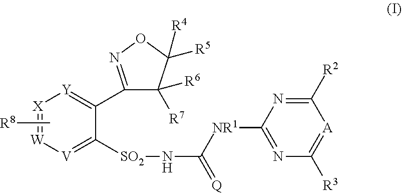
in which
V, W, X and Y are chosen such that one of these indices represents nitrogen and the remaining indices represent carbon atoms, which may be unsubstituted or substituted by the radical R⁸ shown;
A is selected from the group consisting of nitrogen and CR⁹;
where
R⁹ is selected from the group consisting of hydrogen, alkyl, halogen and haloalkyl;
R¹ is selected from the group consisting of hydrogen and an optionally substituted radical from the series consisting of alkyl, alkoxy, alkoxyalkyl, alkenyl, alkynyl, cycloalkyl, cycloalkylalkyl, aralkyl and aryl;
R² is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino;
R³ is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino;
R⁴ to R⁷, in each case independently of one another, are selected from the group consisting of hydrogen, halogen, cyano, alkyl, alkoxy, alkylthio, alkylsulfinyl, alkylsulfonyl, alkylamino, dialkylamino, alkylcarbonyl, alkoxycarbonyl, alkylaminocarbonyl or dialkylaminocarbonyl, it being possible for the radicals to be unsubstituted or to have attached to them one or more radicals selected from the group consisting of halogen, cyano, alkoxy or alkylthio, or R⁴ and R⁶, and R⁵ and R⁷, respectively, represent an alkylidene group which is optionally interrupted by oxygen or sulfur;
R⁸ is selected from the group consisting of hydrogen, halogen, cyano, thiocyanato, nitro, alkyl, alkoxy, alkylthio, alkylsulfinyl, alkylsulfonyl, alkylamino, dialkylamino, alkylcarbonyl, alkoxycarbonyl, alkylaminocarbonyl, or dialkylaminocarbonyl, it being possible for the radicals to be unsubstituted or to have attached to them one or more radicals selected from the group consisting of halogen, cyano, alkoxy and alkylthio;
Q is selected from the group consisting of oxygen or sulfur
and/or salt of a compound of formula (I).

2. An N-azinyl-N′-pyridylsulfonylurea or salt as claimed in claim 1, wherein the substituent A is selected from the group consisting of nitrogen and CH.

3. An N-azinyl-N′-pyridylsulfonylurea or salt as claimed in claim 1, wherein the substituent R¹ is selected from the group consisting of hydrogen, optionally halogen-substituted alkyl, optionally halogen-substituted alkoxy, optionally halogen-substituted alkoxyalkyl, optionally halogen-substituted alkenyl and optionally halogen-substituted alkynyl.

4. An N-azinyl-N′-pyridylsulfonylurea or salt as claimed in claim 1, wherein the substituent R² is selected from the group consisting of hydrogen, halogen, optionally halogen-substituted alkyl, optionally halogen-substituted alkoxy, optionally halogen-substituted alkylthio, optionally halogen-substituted alkylamino and optionally halogen-substituted dialkylamino.

5. An N-azinyl-N′-pyridylsulfonylurea or salt as claimed in claim 1, wherein the substituent R³ is selected from the group consisting of hydrogen, halogen, optionally halogen-substituted alkyl, optionally halogen-substituted alkoxy, optionally halogen-substituted alkylthio, optionally halogen-substituted alkylamino and optionally halogen-substituted dialkylamino.

6. An N-azinyl-N′-pyridylsulfonylurea or salt as claimed in claim 1, wherein the substituents R⁴ to R⁷, in each case independently of one another, are selected from the group consisting of hydrogen, halogen, cyano, thiocyanato, optionally halogen-substituted alkyl, optionally halogen-substituted alkoxy, optionally halogen-substituted alkylthio, optionally halogen-substituted alkylsulfinyl, optionally halogen-substituted alkylsulfonyl, optionally halogen-substituted alkylamino, optionally halogen-substituted alkylcarbonyl, optionally halogen-substituted alkoxycarbonyl and optionally halogen-substituted alkylaminocarbonyl.

7. An N-azinyl-N′-pyridylsulfonylurea or salt as claimed in claim 1, wherein the substituent R⁸ is selected from the group consisting of hydrogen, halogen, cyano, thiocyanato, optionally halogen-substituted alkyl, optionally halogen-substituted alkoxy, optionally halogen-substituted alkylthio, optionally halogen-substituted alkylsulfinyl, optionally halogen-substituted alkylsulfonyl, optionally halogen-substituted alkylamino, optionally halogen-substituted alkylcarbonyl, optionally halogen-substituted alkoxycarbonyl and optionally halogen-substituted alkylaminocarbonyl.

8. A process for the preparation of N-azinyl-N′-pyridylsulfonylurea or a salt as claimed in claim 1, comprising one of the following process steps:
(a) reacting a (4,5-dihydroisoxazol-3-yl)pyridinesulfonamide of formula (II) 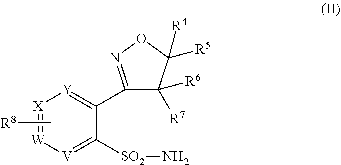
with a heterocyclic (thio)carbamate of the formula (III) 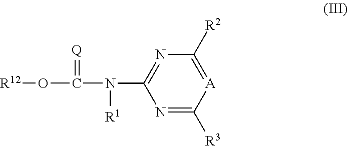
wherein R¹² is a substituted or unsubstituted (C₁-C₂₀)-hydrocarbon radical or
(b) reacting a 4,5-dihydroisoxazol-3-yl)pyridinesulfonyl iso(thio)cyanate of formula (IV) 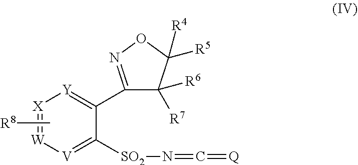
with an aminoheterocycle of the formula (V) 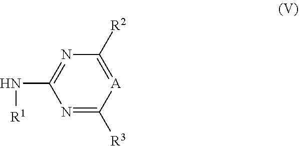
or
(c) reacting a (4,5-dihydroisoxazol-3-yl)pyridinesulfonyl (thio)carbamate of formula (VI) 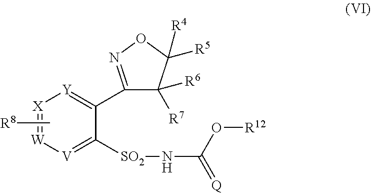
in which R¹² is a substituted or unsubstituted (C₁-C₂₀)-hydrocarbon radical with an amino heterocycle of formula (V) 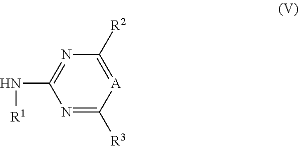
or
(d) reacting a (4,5-dihydroisoxazol-3-yl)pyridinesulfonamide of formula (II) 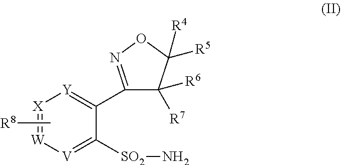
with an iso(thio)cyanate of formula (VII) 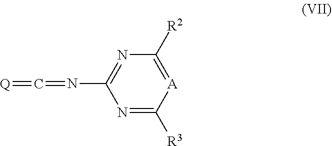
in which R¹ is hydrogen, optionally in the presence of a reaction auxiliary; or
(e) initially reacting, with base catalysis, an amino heterocycle of formula (V) 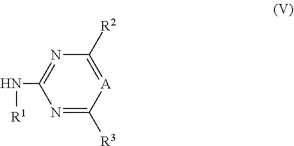
with a carbonic ester and reacting, in a one-pot reaction, the resulting intermediate of formula (III) 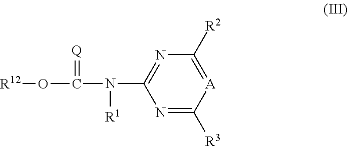
with a (4,5-dihydroisoxazol-3-yl)pyridinesulfonamide of formula (II) 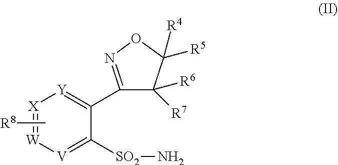
or
(f) reacting a (4,5-dihydroisoxazol-3-yl)pyridinesulfonyl halide of formula (VIII) 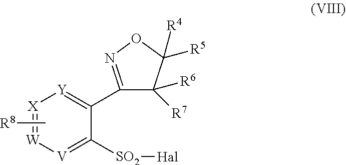
in which Hal is a halogen atom, with a (thio)cyanate to give an iso(thio)cyanate of formula (IV) 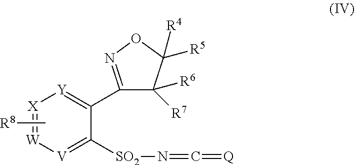
or a solvated (stabilized) derivative, and subsequently with an amino heterocycle of formula (V) 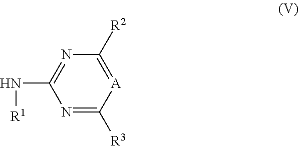
or
(g) reacting a (4,5-dihydroisoxazol-3-yl)pyridinesulfonamide of formula (II) with a heterocyclic biscarbamate of formula (IX) 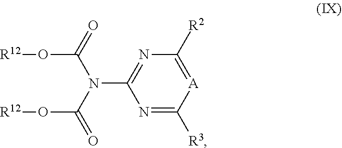
in which R¹² is a substituted or unsubstituted (C₁-C₂₀)-hydrocarbon radical, in the presence of a basic reaction auxiliary, where Q is oxygen or
(h) initially reacting, with base catalysis, a (4,5-dihydroisoxazol-3-yl)pyridinesulfonamide of formula II 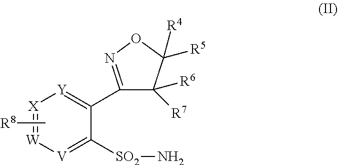
with a carbonic ester and reacting, in a one-pot reaction, the resulting intermediate of formula (VI) 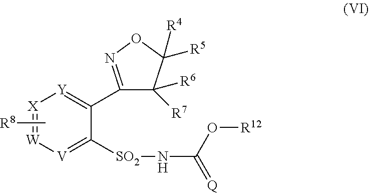
with an amino heterocycle of formula (V)

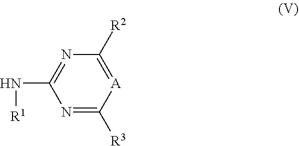

9. A compound of formula (II) 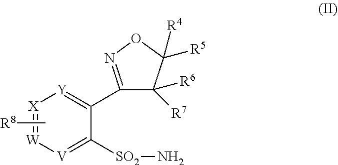
in which the radicals V, W, X, Y, R⁴, R⁵, R⁶, R⁷ and R⁸ have the meanings as claimed in claim 1.

10. A compound of formula (VIII) 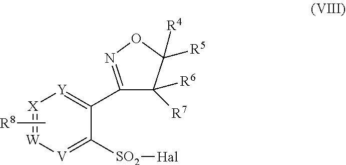
in which the radicals V, W, X, Y, R⁴, R⁵, R⁶, R⁷, R⁸ and Hal have the meanings as claimed in claim 1.

11. A compound of formula (XI) 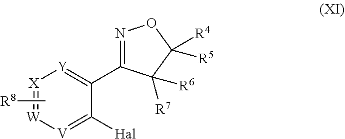
in which the radicals V, W, X, Y, R⁴, R⁵, R⁶, R⁷, R⁸ and Hal have the meanings as claimed in claim 1.

12. A compound of formula (X) 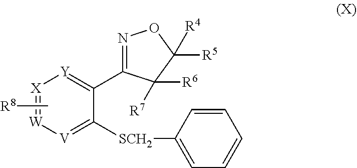
in which the radicals V, W, X, Y, R⁴, R⁵, R⁶, R⁷ and R⁸ have the meanings as claimed in claim 1.

13. A composition comprising at least one compound of formula (I) according to claim 1.

14. The composition as claimed in claim 13, which comprises at least one further active substance which is selected from the group consisting of at least one further herbicide and at least one safener.

15. An herbicide or plant growth regulator comprising a compound of formula (I) as claimed in claim 1.

16. An herbicide or plant growth regulator comprising a composition as claimed in claim 14.

17. A method for controlling plants in specific plant crops or as plant growth regulator comprising using a herbicide or plant growth regulator of claim 15.
